SEQUENCE LISTING (2 sequences)

SEQ ID NO: 1 - protein, 172 aa, Orthinodoros moubata
MNQPDWADEAANGAHQDAWKSLKADVENVYYMVKATYKNDPVWGNDFTCVGVMANDVNEDEKSIQAEFLFMNNADTNMQFATEKVTAVKMYGYNRENAFRYETEDGQVFTDVIAYSDDNCDVIYVPGTDGNEEGYELWTTDYDNILANCLNKFNEYAVGRETRDVFTSACLE

SEQ ID NO: 2 - DNA, 516 bp, Orthinodoros moubata
atgaatcagccagattgggccgatgaagcggcaaatggtgcacaccaagacgcctggaagagtctgaaagcggacgttgaaaacgtttactacatggtgaaggccacctataagaatgacccagtgtggggcaatgacttcacttgcgtgggtgttatggcaaatgatgtcaacgaggatgagaagagcattcaagcagagtttttgtttatgaataatgctgacacaaacatgcaattcgccactgaaaaggtgactgctgttaaaatgtatggttacaatagggaaaacgccttcagatacgagacggaggatggccaagttttcacagacgtcattgcatactctgatgacaactgcgatgtcatctacgttcctggcacagacggaaatgaggaaggttacgaactatggactacggattacgacaacattctagccaattgtttaaataagtttaatgagtacgctgtaggtagggagacaagggatgtattcacaagtgcttgcctagag

(19) United States
(12) Patent Application Publication
(10) Pub. No.: US 2011/0152171 A1
(43) Pub. Date: Jun. 23, 2011

(54) HISTAMINE BINDING PROTEIN

(76) Inventor: Wynne Weston-Davies, London (GB)

(21) Appl. No.: 12/993,815

(22) PCT Filed: May 21, 2009
(86) PCT No.: PCT/GB2009/001284

§ 371 Date: Mar. 7, 2011

(30) Foreign Application Priority Data
May 21, 2008 (GB) 0809279.3

(51) Int. Cl. A61K 38/17; C07K 14/435; C07H 21/00; C12N 15/63; A61P 11/00; A61P 7/02; A61P 9/00; A61P 29/00; A61P 17/06; A61P 9/10; A61P 13/12; A61P 27/02; C12P 21/02; C12N 1/00; C12N 5/10; A61P 37/08; A61P 11/02; A61P 11/08; A61P 19/02; A61P 43/00; A61P 1/00; A61P 1/16; A61P 37/06; A61P 1/02

(52) U.S. Cl. 514/1.5; 530/350; 536/23.5; 435/320.1; 514/21.2; 514/1.8; 514/14.9; 514/16.4; 514/16.6; 514/18.7; 514/1.9; 514/15.4; 514/20.8; 435/69.1; 435/243; 435/325

(57) ABSTRACT

The invention relates to histamine binding proteins. The invention also relates to the use of such histamine binding proteins in the treatment and prevention of diseases.

The present invention relates to a protein that binds to the vasoactive amine histamine and to methods of therapy and diagnosis using these polypeptides.

This application claims priority from GB0809278.3 which is hereby incorporated by reference in its entirety.

### BACKGROUND ART

Vasoactive amines such as histamine and serotonin are mediators of inflammation and regulators of certain physiological processes in animals, including humans. Histamine is present in the secretory granules of mast cells and basophils and is formed by decarboxylation of histidine. It is also present in ergot and plants and may be synthesised synthetically from histidine or citric acid.

The main actions of histamine in humans are stimulation of gastric secretion, contraction of most smooth muscle, cardiac stimulation, vasodilation and increased vascular permeability. In addition to its regulatory role in immune reactions and inflammatory processes, histamine also modulates the production of many cytokines in the body (including those that regulate inflammation) and can interfere with the expression of cytokine receptors. Furthermore, histamine promotes wound healing.

The main pathophysiological roles of histamine are as a stimulant of gastric acid secretion and as a mediator of type I hypersensitivity reactions such as urticaria and hay fever. Histamine or its receptors may also be involved either directly or indirectly in autoimmune disease, e.g. arthritis, and in tumour growth (Falus, 1994).

Histamine produces its actions by an effect on specific histamine receptors which are of four main types, H1, H2, H3 and H4, distinguished by means of selective antagonist and agonist drugs. Histamine H1 and H2 receptor antagonists have clinical uses but at present histamine H3 receptor antagonists are used mainly as research tools.

H1 receptor antagonists (antihistamines) are widely used for treating allergic reactions including allergic rhinitis (hay fever), urticaria, insect bites and drug hypersensitivities. Drugs that lack sedative or muscarinic-receptor antagonist activities are preferred. H1 receptor antagonists are also used as anti-emetics for the prevention of motion sickness or other causes of nausea including severe morning sickness. Muscarinic-receptor antagonist actions of some antihistamines probably contribute to efficacy but also cause side effects. Some H1 receptor antagonists are fairly strong sedatives and may be used for this action.

There are numerous undesirable effects of H1 receptor antagonists. When used for purely antihistamine actions, all the CNS effects are unwanted. When used for their sedative or anti-emetic actions, some of the CNS effects such as dizziness, tinnitus and fatigue are unwanted. Excessive doses can cause excitation and may produce convulsions in children. The peripheral antimuscarinic actions are always undesirable. The commonest of these is dryness of the mouth, but blurred vision, constipation and retention of urine can also occur. Unwanted effects not related to the drugs' pharmacological actions are also seen. Thus gastro-intestinal disturbances are fairly common while allergic dermatitis can follow topical application of these drugs.

H2 receptor antagonists are frequently used as inhibitors of gastric acid secretion. They are used as the drugs of choice in the treatment of peptic ulcer, as second line drugs in the treatment of Zollinger-Ellison syndrome and for treating reflux oesophagitis. Unwanted effects have been reported that include diarrhea, dizziness, muscle pains, transient rashes and hyper-gastrinaemia. Some H2 receptor antagonists can cause gynaecomastia in men and confusion in the elderly.

Besides these unwanted side effects, some histamine antagonists are troublesome if taken with alcohol or with drugs. For example, the antihistamine Seldane used in combination with antibiotics and antifungals may cause life-threatening side effects.

Drugs used to control the actions of histamine are not always effective. The reasons why they may have limited efficacy may relate to the specificity of these drugs for only a subclass of histamine-receptors, particularly when certain conditions require interference with a larger spectrum of receptors. Histamine binding molecules (HBMs) would compete for histamine binding with all receptors and may thus be more suited for treating certain conditions.

There is thus a great need for effective antagonists of histamine that do not generate the side-effects that detract from their applicability to the treatment of human and animal disorders.

It is known that blood-feeding ectoparasites, such as ticks, produce numerous bioactive proteins that immunomodulate the host response to parasite feeding and thereby promote parasite blood-feeding. Such immunomodulatory proteins include histamine binding proteins and examples are presented in granted patents EP-B-0906425 and U.S. Pat. No. 6,617,312. These proteins have shown efficacy as agents for the treatment of allergic rhinitis (see granted patents EP-B-1207899 and U.S. Pat. No. 6,794,360); for the treatment of conjunctivitis (see granted patents EP-B-1207898 and U.S. Pat. No. 6,737,399; and for the treatment of disease conditions mediated by neutrophils (see WO2004/087188).

The protein referred to as FS-HBP2 in EP-B-0906425 has now been studied and further refined for production purposes and to enhance its suitability as a pharmaceutical agent.

### SUMMARY OF THE INVENTION

According to the invention, there is provided a histamine binding protein (HBP) comprising the sequence presented in SEQ ID NO:1. In part, this sequence corresponds to the sequence of FS-HBP2. However, the protein lacks the first 19 amino acids of the FS-HBP2 sequence. The sequence therefore commences at position 20 of the FS-HBP2 sequence. However, additionally, a methionine residue has been appended to the sequence at the N terminus. Furthermore, at position 146 in the amino acid sequence of the protein of the invention, there is a Leucine residue, replacing the Proline residue that occupies position 164 in the amino acid sequence of the FS-HBP2 protein.

The HBP of the invention binds to histamine with high affinity. Preferably, the protein of the invention binds to histamine with a dissociation constant of less than 10⁻⁷ M, more preferably, less than 10⁻⁸ M, less than 10⁻⁹ M or less than 10⁻¹⁰ M.

The HBP of the invention also binds specifically to histamine. The term “specifically” means that the protein has substantially greater affinity for histamine than for other compounds. By “substantially greater affinity” we mean that there is a measurable increase in the affinity for a protein of the invention for histamine as compared with its affinity for other compounds. Preferably, this measurable increase in affinity is at least 10-fold, 100-fold, 10³-fold, 10⁴-fold, 10⁵-fold, 10⁶-fold or greater for histamine than for other compounds. Methods for measuring specificity will be known to those of skill in the art and include competition assays and the like.

The HBP of the invention has been found to be active in the treatment of asthma. This activity has been tested in an acute model of asthma modulation. In this model, clear evidence has been obtained that the HBP of the invention can lower the asthma response. For example, when delivered as an aerosolized treatment, a modulating effect was seen that was similar to that seen with the known asthma modulating agent, budesonide.

However, unlike budesonide, the HBP of the invention does not have any side-effects. No side-effects have been noted in any of the studies reported herein. In contrast, side-effects of budesonide include interactions with ketoconazole (Nizoral), itraconazole (Sporanox), erythromycin (E-Mycin) and many AIDS drugs, as well as headache, nausea, potential psychological changes such as depression and insomnia, also swelling of the face, and loss of bone (osteoporosis).

The inventors have found that using the HBP of the invention, histamine may be almost completely removed from a disease site. In this manner, certain disease conditions may be effectively counteracted. This is only possible using an agent that binds with high affinity to histamine.

This concept is markedly different to that employed by many strategies in the prior art, which target histamine receptors rather than the histamine molecule itself. This can only be effective to the extent that the histamine receptor is blocked, and only then, if it is only that particular receptor that is implicated in the disease. The involvement of any other receptor(s) will not be blocked in this manner and will require additional antagonist agents. Given the degree of redundancy and promiscuity that exists in mammalian systems, it is most unlikely that blockade of a single histamine receptor type will completely prevent the recruitment of neutrophils and other cell types involved in the inflammatory process and this may be one reason for the apparent failure of many histamine antagonists tested so far. In contrast, compounds such as the HBPs of the invention that scavenge free histamine will prevent histamine from reaching any of its receptors, including those that have not yet been discovered. This property contributes to its efficacy as a useful therapeutic agent. Indeed, by combining two approaches of antihistamine and anti-inflammatory in one compound, the HBP of the invention is likely to represent a clinical breakthrough.

The HBP of the invention has been tested in preclinical models of inflammatory, allergic and autoimmune disease and has been demonstrated to be efficacious in this context. In particular, the HBP of the invention reduces the signs of allergen-induced conjunctivitis; it blocks vasoconstriction and reduces airway inflammation in models of asthma; it inhibits eosinophils recruitment in models of asthma; it inhibits neutrophil recruitment and microvascular leakage in models of skin inflammation; it blocks bronchoconstriction in models of ARDS which are resistant to corticosteroids at therapeutic doses; and it down-regulates cytokines IL-4, IL-5, IL-16 and TNFα. The HBP of the invention has also been shown to reduce histamine-stimulated shape change in human eosinophils below baseline levels, whereas specific H4 receptor antagonists and the H3/H4 receptor antagonist thioperamide reduce it to baseline and no further. Histamine stimulated shape change increases the inflammatory potential of eosinophils. The reduction in histamine stimulated eosinophil shape change below base line levels caused by complete removal of histamine by HBP is thus indicative of its enhanced anti-inflammatory activity compared with conventional H4 receptor blocking agents which block a particular receptor.

The HBP of the invention has also been tested in clinical models of allergic rhinitis and allergic conjunctivitis. In allergic rhinitis, the HBP of the invention generated numerical improvement in all five variables that were examined—sneeze, nasal itch, palatal itch, congestion and mucus production. The HBP affects both early- and late-phase symptoms, unlike antihistamines which affect the early stage symptoms and steroids which have a pronounced effect on late-phase symptoms.

The HBP of the invention has also been tested in a study of neutrophil-mediated eye inflammation. The highest dose tested almost totally inhibited the influx of neutrophils into tear fluid. Neutrophil mediated inflammation of the eye is associated with a number of conditions including post-operative cataract surgery, contact lens associated marginal neutrophil infiltration, vernal keratoconjunctivitis and keratoconjunctivitis sicca (dry eye). Post-operative cataract surgery is the most frequently performed surgical procedure in the United States, with more than 1.4 million people having surgery each year. The highest dose tested almost totally inhibited the influx of neutrophils into tear fluid. Particularly in chronic patients HBP represents a potential solution for conditions which are sight threatening.

The HBP of the invention has also been tested in a mouse model of cigarette induced COPD-like inflammation. The protein was found to cause a significant reduction in MMP9 and TIMP-1 protein levels and a marked reduction in TNFα, MIP-2 and keratinocyte chemoattractant levels in the bronchoalveolar lavage fluid (BALF) of smoke-exposed animals. HBP did not affect protein levels of TNFα, keratinocyte chemoattractant, MIP-2, MMP9 or TIMP-1 in sham-treated animals. When the effect of HBP was investigated on the expression of inflammatory cytokines in the lung tissue of smoke-exposed mice, as determined by real-time PCR (inflammatory signature card), HBP caused a reduction in CSF-1, MCP-1, GM-CSF, G-CSF and MIP-2, IL-1β, IL-5, IL-6 and IL-10, p65 and TNFα, TLR2, TREM-1 and e-selectin, and TIMP-1 relative expression levels in the lung tissue of smoke-exposed mice compared to PBS-treated smoke-exposed mice. Furthermore, although neither dexamethasone nor HBP had any effect on myeloperoxidase (MPO) levels in sham animals, cigarette smoke exposure caused a marked increase in MPO levels in lung tissue, which was slightly elevated by subsequent dexamethasone exposure. MPO levels were markedly reduced by HBP. In the same study, the effect of HBP on inflammatory cell recruitment into the lung tissue of smoke-exposed mice was monitored by histological analyses. In sham-treated animals, HBP had no effect on the appearance of the lung, while dexamethasone caused a slight increase in the inflammation induced by PBS instillation in the lungs. This absence of side-effects in comparison to a conventional drug supports the advantageous nature of the HBP of the invention.

The HBP of the invention has been found to be stable. For example, the protein is stable at room temperature (approximately 19° C. to 25° C. or approximately 20° C.). The half-life of the protein is preferably over one hour, preferably over 5 hours, preferably over 10 hours, preferably over 24 hours, more preferably over 48 hours or more, at room temperature. The HBP of the invention has been found to be stable during storage at 4° C. or at a room temperature of 25° C. for at least 52 weeks. Preferably, the half-life of the protein is over one week, preferably over two weeks, preferably over 4 weeks, preferably over 12 weeks, preferably over 26 weeks, preferably over 52 weeks or more at room temperature or at a storage temperature (approximately 4° C.). This facilitates working with the HBP, and makes it easier, for example, for it to be manipulated and administered as a drug to a patient.

Stability of the HBP can be measured by assessing whether it retains molecular integrity over time, for example, whether its molecular weight is altered over time as a result of either degradation or aggregation. This can be assessed by standard methods known in the art, such as SDS-PAGE.

Stability can also be measured by assessing the activity of the protein, since a stable protein preparation will have retained substantially of all of its histamine binding affinity. Ability of the HBP to retain histamine binding activity may be measured directly by detection of HBP-histamine complexes. Alternatively, the ability of the HBP to retain histamine binding activity may be measured indirectly, for example by an assay which measures a change in a cell caused by the reduction in histamine levels due to HBP activity. For example, removal of histamine by HBP decreases histamine-dependent IL-6 production in TNFα activated human umbilical vein endothelial cell (HUVEC) monolayers. The stability of the HBP may therefore be assessed by determining the effect of HBP on IL-6 release by such cells. Preferably, the HBP of the invention inhibits histamine-induced IL-6 production by HUVEC cells by at least 80%, preferably at least 90%, preferably at least 95%, after storage for at room temperature or at a storage temperature (approximately 4° C.) for at least 52 weeks.

The stability of the protein can also be estimated from its sequence, for example, using a bioinformatics tool (ProtParam (ExPASy, Switzerland)). As assessed in this manner, the estimated half-life of the protein is preferably between 20 and 40 hours, more preferably approximately 30 hours in a representative mammalian system (mammalian reticulocytes, in vitro); the estimated half-life of the protein is preferably greater than 10 hours, more preferably greater than 20 hours in yeast in vivo; the estimated half-life of the protein is preferably greater than 5 hours, and more preferably greater than 10 hours in Escherichia coli, in vivo.

The HBP of the invention has been demonstrated to have a half-life in rats of at least 7 hours. Preferably, the HBP has a half-life in a mammal in vivo, preferably a human, of greater than 2 hours, preferably greater than 5 hours, preferably greater than 6 hours, preferably greater than 7 hours. Half-life in vivo may be increased by conjugation or fusion of the HBP to molecules known in the art for this purpose, e.g. polyethylene glycol.

Included as aspects of the invention are functional equivalents of the HBP disclosed herein, such as a natural biological variant, such as an allelic variant or a geographical variant, of a protein with the sequence listed in SEQ ID NO:1; a functional equivalent of a protein with the sequence listed in SEQ ID NO:1 above that contains single or multiple amino-acid substitution(s), addition(s), insertion(s) and/or deletion(s) from the given protein sequence and/or substitutions of chemically-modified amino acids that do not affect the biological function of binding to histamine; and an active fragment of a HBP protein with the sequence listed in SEQ ID NO:1, wherein “active fragment” denotes a truncated protein that retains the biological function of binding to histamine. Excluded from the scope of the invention is the full length FS-HBP2 sequence that is presented in International patent application WO97/44451.

Preferably, the HBP of the invention consists of the amino acid sequence presented in SEQ ID NO:1.

The HBP of the invention may foam part of a fusion protein. For example, it is often advantageous to include one or more additional amino acid sequences which may contain secretory or leader sequences, pro-sequences, sequences which aid in purification, or sequences that confer higher protein stability, for example during recombinant production, or that renders the polypeptide detectable by imaging technology. For instance, a derivative may include an additional protein or polypeptide fused to the HBP at its amino- or carboxy-terminus or added internally to the HBP. The purpose of the additional polypeptide may be to lend additional properties to the HBP as desired. Examples of potential fusion partners include β-galactosidase, glutathione-S-transferase, luciferase, polyhistidine tags, T7 polymerase fragments and secretion signal peptides. Other examples include extracellular domains of membrane-bound proteins, immunoglobulin constant regions (Fc regions), multimerization domains, domains of extracellular proteins, signal sequences, export sequences, and sequences allowing purification by affinity chromatography or sequence allowing imaging, for example fluorescent polypeptides. Other examples will be clear to those of skill in the art. For instance, a polypeptide according to the invention may further comprise a histidine tag, preferably located at the C-terminal of the polypeptide, generally comprising between 1-10 histidine residues, particularly 6 histidine residues.

The HBPs of the present invention can be prepared using known techniques of molecular biology or protein chemistry (for example, chemical peptide synthesis). The HBPs are preferably prepared using the known techniques of genetic engineering as described, for example, by Sambrook et al., Molecular Cloning; A Laboratory Manual, Second Edition (1989), Volumes I and II (D. N Glover ed. 1985); B. Perbal, A Practical Guide to Molecular Cloning (1984); Gene Transfer Vectors for Mammalian Cells (J. H. Miller and M. P. Calos eds. 1987, Cold Spring Harbor Laboratory); Scopes, (1987) Protein Purification: Principles and Practice, Second Edition (Springer Verlag, N.Y.). For example, HBPs of the present invention may be prepared in recombinant form by expression in a host cell. A further aspect of the invention thus provides a method for preparing a HBP of the invention which comprises culturing a host cell containing a nucleic acid molecule according to the invention under conditions whereby said protein is expressed and recovering said protein thus produced. Such expression methods are well known to those of skill in the art and many are described in detail by Sambrook et al., 1989. A suitable expression vector can be chosen for the host of choice. The vector may contain a recombinant DNA molecule encoding a HBP operatively linked to an expression control sequence that is recognized by the host transcription machinery.

Suitable hosts include commonly used prokaryotic species, such as E. coli, or eukaryotic yeasts that can be made to express high levels of recombinant proteins and that can easily be grown in large quantities. Cell lines grown in vitro are also suitable, particularly when using virus-driven expression systems such as the Baculovirus expression system which involves the use of insect cells as hosts. HBPs may also be expressed in vivo, for example in insect larvae or in mammalian tissues.

Preferably, HBP protein is expressed in E. coli; for example, strain BLR(DE3) is suitable. In the embodiment of the invention described in the examples, the protein is expressed from a pET24a-based plasmid (Novagen), although equivalent systems are equally appropriate, as the skilled reader will be aware. The specific protocol described in the Examples is a preferred method for the production of HBPs according to the invention.

According to a yet further aspect, the present invention provides for use of such HBPs to bind histamine in mammals, thereby to regulate its action and to control its pathological effects.

The present invention also includes the use of the HBPs of the present invention as anti-inflammatory agents. In particular, the present invention includes the use of HBPs of the present invention as anti-inflammatory agents for the treatment of late phase or chronic inflammation.

The invention also provides a purified nucleic acid molecule which encodes HBP as described above. Such molecules include DNA, cDNA and RNA, as well as synthetic nucleic acid species. The term “purified nucleic acid molecule” preferably refers to a nucleic acid molecule of the invention that (1) has been separated from at least about 50 percent of proteins, lipids, carbohydrates, or other materials with which it is naturally found when total nucleic acid is isolated from the source cells; (2) is not linked to all or a portion of a polynucleotide to which the “purified nucleic acid molecule” is linked in nature; (3) is operably linked to a polynucleotide which it is not linked to in nature; or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in protein production or its therapeutic, diagnostic, prophylactic or research use. Preferably, the “purified nucleic acid molecule” consists of cDNA only.

Preferably, the purified nucleic acid molecule comprises the nucleic acid sequence as recited in SEQ ID NO: 2 (encoding the HBP of the invention) or is a redundant equivalent or fragment of any one of these sequences. The invention further provides that the purified nucleic acid molecule consists of the nucleic acid sequence as recited in SEQ ID NO: 2 or is a redundant equivalent or fragment of any one of these sequences.

In a further aspect, the invention provides a purified nucleic acid molecule which hybridizes under high stringency conditions with a nucleic acid molecule of the second aspect of the invention. High stringency hybridisation conditions are defined as overnight incubation at 42° C. in a solution comprising 50% formamide, 5×SSC (150 mM NaCl, 15 mM trisodium citrate), 50 mM sodium phosphate (pH 7.6), 5× Denhardts solution, 10% dextran sulphate, and 20 microgram/ml denatured, sheared salmon sperm DNA, followed by washing the filters in 0.1×SSC at approximately 65° C.

In a still further aspect, the invention provides a vector, such as an expression vector, that contains a nucleic acid molecule as described above. Additionally, it may be convenient to cause the recombinant protein to be secreted from certain hosts. Accordingly, further components of such vectors may include nucleic acid sequences encoding secretion signaling and processing sequences. The invention also provides a host cell transformed with such a vector.

Nucleic acid molecules according to the present invention may also be used to create transgenic animals. This may be done locally by modification of somatic cells or by germ line therapy to incorporate heritable modifications. The invention therefore also includes transformed or transfected prokaryotic or eukaryotic host cells or transgenic organisms containing a nucleic acid molecule according to the invention as defined above.

In a further aspect, the invention provides a pharmaceutical composition comprising an HBP, a nucleic acid molecule, a vector, or a host cell according to the aspects of the invention described above, in conjunction with a pharmaceutically-acceptable carrier. Preferably, such pharmaceutical compositions comprise HBPs according to the invention, optionally including an inert carrier or carriers. The HPB may constitute the sole active component of the composition or can form part of a therapeutic package, such as a component of a cream, aerosol or aqueous composition. In the case of an aqueous composition, this can of course be lyophilised for distribution, and the lyophilised material can eventually be reconstituted with an aqueous carrier for administration to patients. Thus any process for the preparation of a composition according to the invention may further comprise the steps of lyophilising the composition and then, optionally, reconstituting the composition with an aqueous medium.

In one preferred embodiment, the HBP of the invention is formulated in a formulation buffer, 12.6 mM Sodium Phosphate, 124 mM Sodium Chloride, pH 7.2. The protein is then serially diluted from this stock in PBS.

In another preferred embodiment, the HBP of the invention may be formulated as a cream, preferably a water-based cream.

In another preferred embodiment, the HBP of the invention may be an aerosol, preferably comprising dry powder with lactose, with HFA (hydrofluoroalkane) as propellant.

Once formulated, the compositions of this aspect of the invention can be administered directly to the subject. The subjects to be treated can be animals; in particular, human subjects can be treated.

The invention also provides a method for treating a patient, comprising administering a pharmaceutical composition of the invention to the patient. The patient is preferably a human, and may be a child (e.g. a toddler or infant), a teenager or an adult, but will generally be an adult. The invention also provides HBP compositions of the invention for use as a medicament.

The invention also provides the use of HBPs and other compositions of the invention in the manufacture of a medicament for treating a patient. These uses, methods and medicaments are preferably for the treatment of a condition in which histamine has a role. Such conditions include allergies, such as allergic rhinitis, allergic conjunctivitis (including severe allergic conjunctivitis), vernal keratoconjunctivitis (VKC), diffuse lamellar keratitis, infective and non-specific conjunctivitis, keratitis and blepharitis; and disease conditions in which neutrophils are implicated, including adult respiratory distress syndrome (ARDS); infant respiratory distress syndrome (IRDS); severe acute respiratory syndrome (SARS); chronic obstructive airways disease (COPD); cystic fibrosis; ventilator induced lung injury (VILI); capillary leak syndrome; reperfusion injury including injury following thrombotic stroke, coronary thrombosis, cardiopulmonary bypass (CPB), coronary artery bypass graft (CABG), limb or digit replantation, organ transplantation, bypass enteritis, bypass arthritis, thermal injury and crush injury; psoriasis; psoriatic arthropathy; rheumatoid arthritis; Crohn's disease; ulcerative colitis; immune vasculitis including Wegener's granulomatosis and Churg-Strauss disease; alcoholic liver disease; neutrophil mediated glomerulonephritis; systemic lupus erythematosus; lupus nephritis; atherosclerosis; systemic sclerosis; gout; periodontal disease, ocular inflammation including dry eye, Sjogren's syndrome, contact lens associated papillary conjunctivitis (CLAPC), contact lens associated marginal infiltrates, post surgical inflammation including surgery for cataract, glaucoma, corneal transplantation and laser in-situ keratomileusis (LASIK), shield ulcers; osteoarthritis; wet and dry age related macular degeneration (AMD); macular oedema including post-operative cystoid macular oedema (CME); malignant disease including carcinoma of the breast and malignant melanoma; anaphylaxis and severe allergy including peanut and latex allergy; irritable bowel syndrome (IBS); and interstitial cystitis.

Compositions according to the invention should be administered directly to a patient in a therapeutically effective amount. The term “therapeutically effective amount” as used herein refers to an amount of HBP needed to treat, ameliorate, or prevent the targeted disease condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

Direct delivery may be accomplished by parenteral injection (e.g. intravenously, subcutaneously, intraperitoneally, intramuscularly, or to the interstitial space of a tissue), or by rectal, oral, vaginal, topical, transdermal, intranasal, ocular, aural, pulmonary or other mucosal administration. The precise mode of administration will depend on the disease or condition to be treated. For example, to treat the symptoms of allergic rhinitis, HBPs according to the invention may be inhaled, for example, as an aerosol.

HBP dosing is usually scaled to a patient's body size, measured either by body weight (kg) or by body surface area (BSA; measured in m², measured, or estimated by a combination of a patient's height and weight). Although there is no exact conversion between weight and BSA dosing, there is a good approximation: for a person of average weight and height (50th percentile for each), 25000 IU/kg=1 MIU/m².

Treatment can be a single dose schedule or a multiple dose schedule. A typical treatment regimen for the HBPs of the invention as used in asthma is to administer between 5 and 10 μg/kg per 24-hours. Preferably, this is administered in two doses. An exemplary dosage is 7.2 μg/kg per 24-hours. Extrapolating from other drugs used for both asthma and rhinitis it is likely that the effective dose in asthma will be 2-4 times that i.e. between 10 and 40 μg/kg per 24-hours, preferably between 15 and 30 μg/kg per 24-hours.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.005 mg/kg to 50 mg/kg, preferably 0.125 mg/kg to 20 mg/kg. For example, particularly preferred dosages of the HBP referred to herein are between 0.1 to 20 mg/kg, more preferably, 0.5 to 10 mg/kg, still more preferably 1 to 2 mg/kg. Compositions may be administered individually to a patient or may be administered in combination with other agents, drugs or hormones.

Preferred administrations include injection, inhalation, intravenous administration, intraperitoneal administration and topical administration. In particular, preferred routes include inhalation by aerosol for allergic rhinitis; topical application by eye drop for allergic conjunctivitis and allergic rhinitis; topical application by cream for skin rash allergies; nasally for allergic rhinitis; orally as an aqueous solution or dispersion for Crohn's disease; infusion for treatment of malignant melanoma, rheumatoid arthritis etc.

HBPs of the invention can be used as the active ingredient of pharmaceuticals. Such pharmaceuticals can be used on their own to treat patients, or can be used in conjunction with other active ingredients. Typically, the HBPs will not be mixed with any other active ingredient before administration; rather, the HBP and other active ingredient(s) will be administered as separate independent medicines in a combined protocol. Many of the ascribed indications are customarily treated by combination therapy. Thus the invention provides (a) HBPs of the invention, and (b) a second pharmaceutical agent, for simultaneous separate or sequential administration. The invention also provides a pharmaceutical preparation or system, comprising (a) a first pharmaceutical agent, which comprises HBPs of the invention; and (b) a second pharmaceutical agent, wherein said first and second agents are either in admixture or are separate compositions e.g. for simultaneous separate or sequential administration.

The invention also provides a kit comprising (a) a first pharmaceutical agent, which comprises HBPs of the invention; and (b) a second pharmaceutical agent. Examples of the second pharmaceutical agent include histamine blocking agents, and H1, H2, H3 and/or H4 receptor antagonists.

Combination therapy is particularly applicable to malignant disease and asthma. Examples of second pharmaceutical agents for malignant disease include alkylating agents such as cyclophosphamide; cytotoxic antibiotics such as bleomycin; antimetabolites such as methotrexate; vinca alkaloids such as vincristine; antibodies such as cetuximab; platinum compounds such as cisplatin; taxanes such as paclitaxel; topoisomerase inhibitors such as Trastuzumab; hormone antagonists such as tamoxifen; anti-androgens such as buserelin and somatostatin analogues such as ocreotide.

For asthma second pharmaceutical agents would include: beta adrenergic bronchodilators such as salbutamol; antimuscarinic bronchodilators such as ipratropium bromide; mast cell stabilising agents such as sodium cromoglycate; theophylline derivatives such as aminophylline; leukotriene antagonists such as montelukast; immunoglobulin E antibodies such as omalizumab; and phosphodiesterase inhibitors such as roflumilast.

The invention also provides the use of (a) HBPs of the invention and (b) a second pharmaceutical agent, in the manufacture of a combination medicament.

The invention also provides the use of HBPs of the invention in the manufacture of a medicament, wherein the medicament is for administration to a patient who has been pre-treated with a second pharmaceutical agent. Similarly, the invention provides the use of a second pharmaceutical agent in the manufacture of a medicament, wherein the medicament is for administration to a patient who has been pre-treated with HBPs of the invention. The pre-treatment may be recent (e.g. within the 24 hours preceding administration of said medicament), intermediate (e.g. more than 24 hours previous, but no longer than 4 weeks), more distant (e.g. at least 4 weeks previous), or very distant (e.g. at least 6 months previous), with these time periods referring to the most recent pre-treatment dose. The patient may be refractory to treatment by the pharmaceutical agent that was administered in the pre-treatment.

Gene therapy may be employed to effect the endogenous production of a histamine binding protein by specific cells in a patient. Gene therapy can either occur in vivo or ex vivo. Ex vivo gene therapy requires the isolation and purification of patient cells, the introduction of the therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a histamine binding protein may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic histamine binding compound is directly injected into the bloodstream or muscle tissue.

The invention also provides the use of HBPs of the invention in the manufacture of a medicament, wherein the medicament is co-administered with a second pharmaceutical agent. Similarly, the invention provides the use of a second pharmaceutical agent in the manufacture of a medicament, wherein the medicament is co-administered with HBPs microaggregates of the invention. The two agents are preferably administered within 4 hours of each other.

The present invention also includes the use of HBPs as tools in the study of inflammation, inflammation-related processes or other physiological effects of vasoactive amines such as the role of histamine in the formation of gastric ulcers or its role in immune reactions. For example, the HBPs may be used for histamine depletion in cell cultures or in inflamed animal tissues in order to study the importance of histamine or serotonin in these systems.

Various aspects and embodiments of the present invention will now be described in more detail, with particular reference to the HBP whose sequence is presented in SEQ ID NO:1. It will be appreciated that modification of detail may be made without departing from the scope of the invention.

### BRIEF DESCRIPTION OF THE FIGURES

FIG. 1 shows penH values against increasing doses of methacholine.

FIG. 2 shows numbers of cells within the BAL of experimental mice.

FIG. 3 shows graphs of total viable cells, macrophages, neutrophils and lymphocytes in BALF of sham or smoke exposed mice after treatment with PBS, steroid or HBP.

FIG. 4a shows graphs of protein levels of TNFα, MIP-2 and KC in BALF of sham or smoke-exposed mice after treatment with PBS, steroid or HBP. 4b: Graphs of protein levels of MMP9 and TIMP-1 in BALF of sham or smoke-exposed mice after treatment with PBS, steroid or HBP.

FIG. 5 shows graphs of MMP2 and MMP9 levels and protease activity in BALF of sham or smoke-exposed mice after treatment with PBS, steroid or HBP.

FIG. 6a shows graphs of the relative expression of monocyte chemattractant factors in lungs of sham or smoke-exposed mice after treatment with PBS, steroid or HBP. 6b: Graphs of the relative expression of interleukins in lungs of sham or smoke-exposed mice after treatment with PBS, steroid or HBP. 6c: Graphs of the relative expression of NF-kappa B subunits and TNFα in lungs of sham or smoke-exposed mice after treatment with PBS, steroid or HBP. 6d: Graphs of the relative expression of cell adhesion and signaling factors in lungs of sham or smoke-exposed mice after treatment with PBS, steroid or HBP. 6e: Graphs of the relative expression of proteases and anti-proteases in lungs of sham or smoke-exposed mice after treatment with PBS, steroid or HBP.

FIG. 7 shows a graph of the level of myeloperoxidase in lungs of sham or smoke-exposed mice after treatment with PBS, steroid or HBP.

FIG. 8a shows histological images of the lungs of sham mice after treatment with PBS, steroid or HBP. 8b: Histological images of the lungs of smoke-exposed mice after treatment with PBS, steroid or HBP.

FIG. 9 shows the effect of HBP on histamine stimulated eosinophil shape change. Histamine concentration 0.5 μM. HBP used at 10 mg (A), 7.5 mg (B), 5.0 mg (C), and 2.5 mg (D).

### EXAMPLES

### Example 1

### HBP Activity Tested in an Acute Model of Asthma Modulation

The aim of the following test is to ascertain the response to HBP at three different concentrations in OVA-sensitised and challenged mice, compared with Budesonide treated and unsensitised/unchallenged controls.

Methodology

Protocol:

The work tested the response to the HBP of the invention in OVA sensitised and challenged mice. The HBP substance lot number 430-1105-003 at 9.84 mg/ml was used, produced by Evolutec. The sequence of the HBP protein is provided in SEQ ID NO:1. The coding sequence is provided in SEQ ID NO:2.

HBP protein is expressed from a pET24a-based plasmid in E. coli strain BLR(DE3). For production of the HBP protein, 10 shake flasks containing 1.0 L media each are inoculated. Shake flasks are then incubated at 37° C. and 200 rpm. During growth, the culture OD₆₀₀ is monitored in a single flask, termed Shake flask #1. When the OD₆₀₀ of Shake flask #1 reaches 2.0±0.5, the contents of the other 9 flasks are combined and used to inoculate 1000 L of fermentation media in a 1500 L fermentor (1200 L working volume). An ECPM1-based fermentation medium using glycerol and yeast extract as the primary carbon, energy and nitrogen sources is used for HBP production. Kanamycin is added to a final concentration of 50 μg/ml. The medium is formulated with animal-free components and supports growth to high cell densities.

A complex feed medium consisting of concentrated glycerol and yeast extract is fed to the batch culture at a linear rate of 2 mL/min starting once the batch culture reaches an OD₆₀₀ₙₘ of 10 to 15. HBP product induction is initiated by addition of IPTG to a final concentration of 0.2 mM once the culture reaches mid-exponential phase (OD₆₀₀ₙₘ=40 to 50). Induction is allowed to proceed for two hours after which harvest operations are initiated. Foaming is monitored visually and antifoam (Pluronic L61 Surfactant) is added as required.

Cells are harvested from the fermentor and washed using tangential flow filtration (TFF) in a Uniflux 400 filtration unit. Upon completion of the harvest, the cell slurry is aliquoted 20 kg per bag into 20 L media bags and frozen as whole cell slurry prior to downstream processing. Generally a 1000 L fermentation is expected to yield approximately 200 kg total cell slurry. HBP is expressed at high levels in intracellular inclusion bodies, and is obtained from the cells through homogenization, solubilization of the protein, and refolding. The cells are thawed, lysed by 2 passes through the homogenizer, and the inclusion bodies are isolated by TFF. The protein contained within the inclusion bodies is solubilized and refolded in a single step refold procedure with 20-fold dilution.

Following refold, the protein is purified using two chromatographic steps. The first purification is the capture column Q-sepharose FF, an anion exchange resin. The pooled HBP-containing fractions from the Q-Sepharose FF column are then loaded onto a Butyl 650S HIC column for further purification and polishing. Following elution from the Butyl 650S and pooling of the appropriate fractions, the product is diafiltered and concentrated into the final formulation buffer, 12.6 mM Sodium Phosphate, 124 mM Sodium Chloride, pH 7.2. The diafiltered retentate is tested for Endotoxin and protein concentration. If the Endotoxin level in the eluate is >10 EU/mg HBP, the diafiltered retentate is filtered through a Mustang E filter and retested for Endotoxin. The final product is filtered through a 0.2 μ filter and aliquoted.

The treatments were made via serial dilution with PBS by KWS Biotest Ltd., UK.

Animals: 48 female Balb/c mice bred within the KWS breeding unit and used at 7 to 9 weeks of age.

Component 1: Clinical Endpoints

Six groups of 8 mice were used aged 7-9 weeks of age. Mice in groups B-F were sensitised to OVA by injection i.p. with OVA in alum day 0 and day 14. All animals were then challenged by aerosol exposure to 5% OVA in distilled water for 20 minutes daily from day 18-23. In addition mice in groups C-F were given treatments by aerosolisation on days 21-24 as described below 1 hour prior to OVA challenge. At termination (day 24) all animals were exposed to increased concentrations of methacholine from 3.125 mg/ml to 50mg/ml in PBS. Bronchoalveolar lavage (BAL) fluids were also collected for preparation of cytospins for analysis of infiltrating inflammatory cells.

Treatment Groups (n=8)

A—Unsensitised/challenged/untreated

B—Sensitised/challenged/untreated

C—Sensitised/challenged/treated HBP at 75 μg per mouse per day

D—Sensitised/challenged/treated HBP at 150 μg

E—Sensitised/challenged/treated HBP at 300 μg

F—Sensitised/challenged/treated budesonide ling in PBS

Preparation of antigen for sensitisation: Alum precipitated Ova (chicken OVA, grade V, SIGMA) was prepared by;
1. Dissolve 20 mg Ova in 1 ml PBS.
2. mix with 4.6 ml of sterile (filtered) 8.4% NaHCO3 (0.42 g in 5 ml H₂O) and placed on a stirrer.
3. Ten ml of sterile (filtered) 9% aluminium potassium sulphate (Alum) was added dropwise (by 10 ml syringe with needle).
4. Spin down at 3000 rpm in a bench centrifuge for 1 minute.
5. Wash the pellet twice by in 0.9% sodium chloride (saline) followed by centrifugation.
6. Resuspend to give a final volume of 10 ml (2 mg/ml) in saline and store at 4° C.
7. Before inoculation the alum precipitated OVA was diluted 1:40 in saline to give 50 μg/ml OVA in Alum.
8. Inject mice with 200 μl i/p day 0 and day 14.

Preparation of Treatments
1. HBP stock at 9.84mg/ml (Solution A) require 20 mls at 400 μg/ml.
2. Dilute 813 μl stock in 20 mls PBS (Solution B)
3. Add 3 mls B to 9 mls PBS to give 100 μg/ml (Solution C) use 6 mls in nebuliser expose mice for 20 mins—Group C
4. Add 6 mls B to 6 mls PBS to give 200 μg/ml (Solution D) use 6 mls in nebuliser expose mice for 20 mins—Group D
5. Use Solution B neat in nebuliser as above—Group E
6. Dissolve 250 mg Budesonide in 2.5 mls 70% absolute ethanol to give 100 mg/ml. Dilute, on day, 10 μl in 6 mls PBS, nebulise mice 20 mins as above—Group F

Procedure:
1. Day 0—sensitise mice groups B-F to ovalbumin by administration of 10 μg in 200 μl of OVA in alum i/p. Require sufficient for 40 mice (8 ml) therefore make up 10 ml by adding 250 μl stock to 9.75 ml PBS.
2. Day 14—repeat above.
3. Day 18-23 challenge all mice by nebulisation of 5% OVA diluted in sterile DW for 20 mins (Pari LC Star Nebuliser). Set apparatus up to anaesthetic box, ensure gas can escape, add approximately 6 mls 5% sterile filtered OVA solution. Run for 20 mins.
4. Day 21-24 treat mice in groups C-F 1 hour prior to challenge with OVA as described above
5. Day 24—Measure airway reactivity (AR) using Buxco machine.
   Collect BAL fluids for cytospin and analysis of infiltrate.
   Retain supernatant at −20° C. for future cytokine analysis.
   Remove lung into 10% buffered formalin for histopathology

Disease: Assess hyperresponsiveness following sensitisation.

Clinical Disease Score:
1. Buxco penH readings (see http://www.buxco.com/Response%20Standard.pdf)
2. Analysis of infiltrating cells by Leishmans staining of cells isolated from BAL fluids. A minimum of 5× fields of view were counted/slide (×100 oil immersion).

Results

The primary endpoint used in the study was airway hyper-responsiveness as assessed using penH (see http://www.buxco.com/Response%20Standard.pdf). The algorithm for Penh is derived from whole body plethysmography experiments and compares the average amplitude of the early part of the expiratory phase to the average amplitude of the later part of the expiratory phase; and the peak amplitude of the expiratory phase to the peak amplitude of the inspiratory phase.

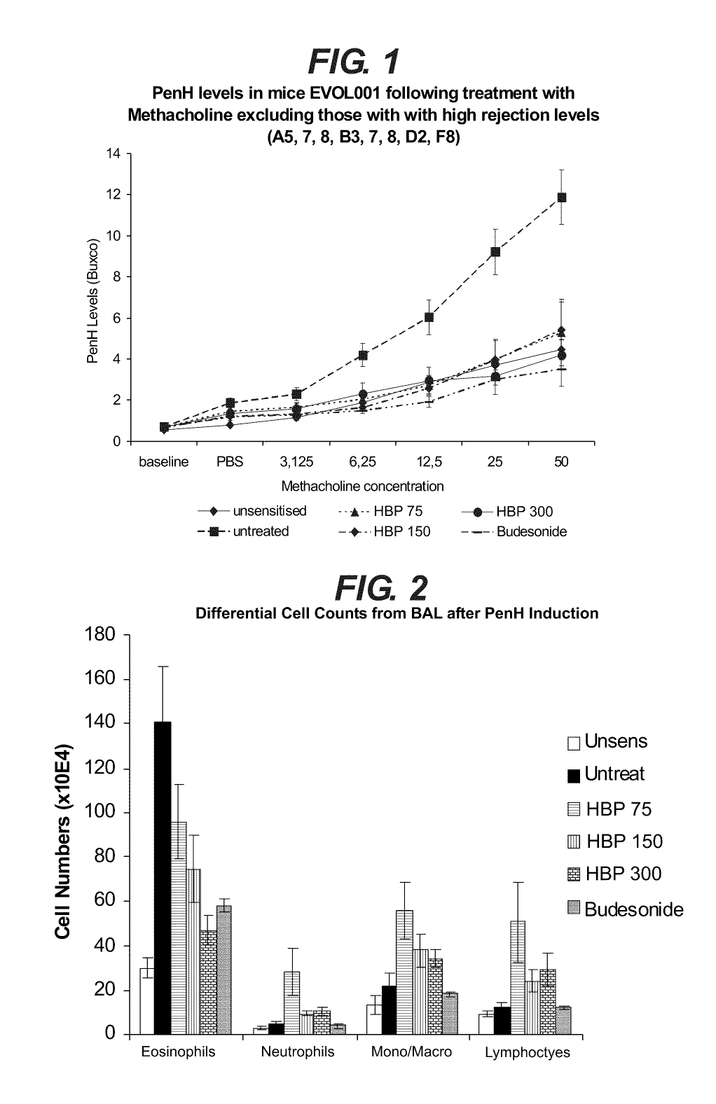

FIG. 1 shows the penH values. The data show that sensitisation led to a marked increase in airway responsiveness to methacholine challenge. As expected, Budesonide treatment markedly reduced the penH response. Similarly, all three test doses of HBP reduced the penH response in treated animals. There was very little effect of dose between the 75, 150 and 300 μg doses of HBP tested.

The secondary endpoint used in the study was infiltration of the BAL with inflammatory cells. The data of this analysis are shown in FIG. 2. The data show a clear difference in the numbers of eosinophils in the BAL fluids of sensitised versus unsensitised animals, validating the data set. Sensitisation caused only very minor increases in the number of neutrophils, macrophages and lymphocytes within the BAL fluids. The lower level increases in the numbers of these cell types is expected within the model, although in analogous experiments slightly larger increases are often seen. Budesonide lowered the numbers of eosinophils to near background levels. Numbers of neutrophils, macrophages and lymphocytes were similar between budesonide, unsensitised and untreated controls.

Treatment with HBP reduced eosinophil numbers in BAL fluids, in keeping with its effects on penH values following methacholine exposure. In contrast to the penH data, there was a clear dose effect. 75 μg HBP lowered eosinophil numbers only partially, 150 μg produced a larger decrease, and 300 μg HBP reduced numbers of eosinophils to levels equivalent to those seen after budesonide treatment. The data suggest that while HBP decreased numbers of eosinophils in the BAL, numbers of neutrophils, macrophages and lymphocytes were slightly though, not significantly elevated in comparison to untreated controls. The lack of significance of this increase together with the inverse dose response suggests that this is most likely accounted for by the relatively low numbers of these cells observed in the BAL of untreated mice.

### CONCLUSION

The data provide clear evidence that HBP can lower the asthma response in an acute model. Aerosolised treatment is able to produce a modulating effect similar to that seen with a known asthma modifying agent, budesonide. The ability of HBP to modify the response was more apparent when looking at airway hyper-responsiveness than when looking at BAL cell numbers. The maximal clinical effect was achieved with the lowest dose of HBP used, whereas this dose produced only a very mild decrease in the numbers of eosinophils in the BAL.

### Example 2

### Comparative Effect of the HBP Protein and a Steroid (Dexamethasone) on a Range of Parameters in a Mouse Model of Cigarette Induced COPD-Like Inflammation.

2.1. Study Design

2.1.1 Objectives: To assess the effect of the HBP protein and a steroid (dexamethasone) on a range of parameters in a mouse model of cigarette induced COPD-like inflammation.

2.1.2 Group Size: n=8

2.1.3 Protocol: Male Balb/c mice 6-8 weeks old were exposed to cigarette smoke (9 Winfield cigarettes per day with <16 mg tar, <1.2 mg/kg nicotine and <15 mg CO) for 4 days, 15 min exposure per cigarette and then dissected on day 5.

2.1.4 Groups: Sham+HBP test drug
Sham+placebo (PBS)
Sham+steroid comparator
Smoke+HBP test drug
Smoke+placebo
Smoke+steroid comparator

2.1.5 Drug Formulation:

Test Compound HBP:

Dosage: 10 mg/mL, i.p. (administered one hour prior to first smoke exposure each day).

Solvent: Phosphate Buffered Saline

Steroid Comparator, Dexamethasone:

Dosage: 1 mg/kg, i.p. (administered one hour prior to first smoke exposure each day).

Solvent: sterile MilliQ water

| | | |
|---|---|---|
| 2.1.6 Endpoints: | BAL fluid - | total/differential cell counts |
| | | ELISAs (TNF alpha,keratinocyte |
| | | chemoattractant, TIMP-1, MIP-2 and |
| | | MMP9) |
| | | zymography (protease induction) |
| | | myeloperoxidase assay |
| | Lung | PFA fixation and histology (4 mice per |
| | | group) |
| | | inflammatory signature card (gene |
| | | analyses) |
| | Muscle | tissues reserved for possible future |
| | | metabolic |
| | | profiling |

2.1. 7 Data Collection: The Following Parameters Will be Monitored Throughout The Study In All Groups:
General clinical observations for all groups

2.2. Methods

2.2.1 Animals

Specific pathogen-free male Balb/C mice aged 7 weeks and weighing ˜20 g were obtained from the Animal Resource Centre Pty. Ltd. (Perth, Australia). The animals were housed at 20° C. on a 12-h day/night cycle in sterile micro-isolators and fed a standard sterile diet of Purina mouse chow with water allowed ad libitum.

2.2.2 Cigarette Smoke Exposure

Mice were placed in an 18 litre perspex chamber in a class II biosafety cabinet and exposed to cigarette smoke as described. Mice were exposed to cigarette smoke generated from 9 cigarettes per day for 4 days, delivered three times per day at 8.00 am, 12 noon and 4 pm using 3 cigarettes spaced over one hour. In pilot experiments we found that 3, 6 and 9 cigarettes per day are very well tolerated. Sham-exposed mice were placed in an 18 litre perspex chamber but do not receive cigarette smoke. On the fifth day, mice were killed by an intraperitoneal (i.p.) overdose of anaesthetic (5.6 mg ketamine/1.12 mg xylazine, Parnell Laboratories, NSW, Australia) and the lungs lavaged with PBS as described later. Commercially available filter-tipped cigarettes (manufactured by Philip Morris, Australia) of the following composition were used: 16 mg or less of tar, 1.2 mg or less of nicotine and 15 mg or less of CO. Smoke was generated in 50 ml tidal volumes over 10 s using timed draw-back mimicking normal smoking inhalation volume and cigarette bum rate. Group sizes of 8 mice per treatment were used to ensure the study was powered to detect differences in response variable at the 0.05 confidence level.

2.2.3 Drug Administration

Mice were given the specified doses of test drug, placebo (PBS) or dexamethasone (as outlined in section 1) once daily (60 minutes prior to first smoke), administered by i.p. injection.

2.2.4 Bronchoalveolar Lavage (BAL)

BAL was performed in terminally anaesthetised mice. Briefly, lungs from each mouse were lavaged in situ with a 400 μl aliquot, followed by three 300 μl of PBS, with approximately 1 ml of bronchoalveolar lavage fluid (BALF) recovered from each animal. Smoke exposure had no effect on the recovered volume. The total number of viable cells in the BALF was determined by using the fluorophores ethidium bromide and acridine orange (Molecular Probes, San Diego, USA) on a standard Neubauer hemocytometer using a Zeiss Axioscope Fluorescence microscope. Cytospins were prepared using 200 μl BALF at 350 rpm for 10 min on a Cytospin 3 (Shandon, UK). Cytospin preparations were stained with DiffQuik (Dade Baxter, Australia) and cells identified and differentiated into mononuclear, epithelial, eosinophils, neutrophils and macrophages by standard morphological criteria. Mitotic figures, an index of cell division, were identified by standard morphological criteria. A minimum of 500 cells per slide were counted.

2.2.5 Enzyme Linked Immunosorbant Assays (ELISAs)

TNFα, keratinocyte chemoattractant (KC), MIP-2, MMP9 and TIMP-1 concentrations in BALF samples were measured using Pharmingen OptEIA™ ELISA kits (Pharmingen) as per manufacturer's instructions. The absorbances were read at 450 nm (Victor 1420 Multilabel Counter, Wallac), and analysed using the Microplate Manager® (BioRad, USA) program, which derived the standard curve and sample absorbances.

2.2.6 Protease Expression and Activity in BALF

Zymography was used to assess protease expression in response to cigarette smoke exposure. Briefly, BALF from animals in each treatment group were pooled and concentrated by adding 250 μl of 50% trichloroacetic acid to 500 μl of pooled BALF samples and left at 4° C. overnight. The next day samples were spun (13,000 rpm for 10 min, at 4° C.) and the pellet washed twice with 300 μl 80% diethyl ether (in 20% ethanol) and dried in air for 10 min. The pellet was then resuspended in 50 μl of 1× non-reducing buffer, heated for 10 min at 65° C. and 20 μl loaded on SDS-page mini-gels. SDS-page mini-gels (10%) were prepared with the incorporation of gelatin (2 mg/ml) before casting. BALF (20 μl) was run into gels at a constant voltage of 200 V under non-reducing conditions. When the dye front reached the bottom, gels were removed and washed twice for 15 min in 2.5% Triton X-100 and incubated at 37° C. overnight in zymography buffer (50 mM Tris-HCl (pH 7.5), 5 mM CaCl₂, 1 mM ZnCl₂ and 0.01% NaN₃). The gels were then stained for 45 min with Coomassie Brilliant Blue R-250 and extensively destained. Following destaining, zones of enzyme activity appeared clear against the Coomassie Blue background.

Neat BALF was also tested for net gelatinase and net serine protease activity using fluorescence-conjugated gelatin (Molecular Probes, USA) and N-methoxysuccinyl-ala-ala-pro-val-p-Nitroanilide (Sigma, USA), respectively. The gelatin substrate (10 μg) was diluted in 50 mM Tris pH 7.5, 150 mM NaCl, 5 mM CaCl₂, 0.01% NaN₃ and incubated at room temperature for 16 h with 100 μl of neat BALF. The digested substrate had absorption/emission maxima at 495 nm/515 nm. The N- methoxysuccinyl-ala-ala-pro-val-p-Nitroanilide substrate (50 μg) was diluted in 50 mM Tris pH 7.5, 150 mM NaCl, 5 mM CaCl₂, 0.01% NaN₃ and incubated at room temperature for 16 h with 100 μl of neat BALF. The digested substrate had absorption maxima at 405 nm. The fluorescence intensity of the substrates was measured in a microplate reader (Victor II, Wallac) to detect quantitative differences in activity.

2.2.7 RNA Extraction and Quantitative Real-Time PCR

Whole lungs were perfused free of blood via right ventricular perfusion with 10 ml warmed saline, rapidly excised en bloc, blotted and snap frozen in liquid nitrogen. Total RNA was isolated from 15 mg of whole lung tissue according to manufacturer instructions using the RNeasy kit (Qiagen). The purified total RNA prep was used as a template to generate first-strand cDNA using SuperScript II (Invitrogen). The reaction mix containing 1 μg of RNA, 250 ng of random hexamers (Promega) and 10 mM dNTP mix was made up to 12 μl with sterile water, heated to 65° C. for 5 min and chilled on ice for 1 min. First strand synthesis was then performed in 20 μl of total reaction volume by adding 50 mM Tris.HCl (pH 8.3), 75 mM KCl, 3 mM MgCl₂, 10 mM DTT, 40 U RNaseout and 200 U Superscript II reverse transcriptase enzyme at 42° C. for 50 min followed by enzyme inactivation at 70° C. for 15min. cDNA was diluted 10-fold in sterile water and stored at −20° C. prior to amplification.

Quantitative real-time PCR technique based on the 5′ exonuclease activity of the Taq polymerase was used. In addition to the sense and antisense primer, an oligonucleotide probe with a 5′ fluorescent reporter dye (6 FAM) and a 3′ quencher dye (TAMRA) hybridized downstream of the sense primer to the target sequence. Based on a 10 μl reaction volume performed in a 384 optical well plate, the master mixture was prepared from the TaqMan Universal Master Mix (Applied Biosystems) comprising of AmpliTaq Gold DNA polymerase, Amperase UNG, dNTPs (dCTP, dGTP, dATP, and dUTP), passive reference 6-carboxy-rhodamine (ROX), MgCl_{2,} and buffer components in amounts undisclosed by the manufacturer.

2.2.8 General Use of Microfluidic (Inflammatory Signature) Card

To maximise accuracy and comparability pre-optimized primers and probes were purchased from Applied Biosystems and custom configured in microfluidic card format. As an internal control, eukaryotic 18S rRNA (Applied Biosystems) was measured for use as a reference. A negative (no-template) control was included in every run. The fluorescence signal was monitored on-line using the laser detector of the ABI Prism 7900 HT Sequence Detection System (Applied Biosystems) under default cycling parameters for the microfluidic card format. Each assay was performed in replicates of four. The cycle threshold (C_{T}) value was the PCR cycle number (out of 40) at which the measured fluorescent signal exceeded a calculated background threshold identifying amplification of the target sequence value and was proportional to the number of input target copies present in the sample. Threshold cycle numbers were transformed using the ΔΔCt (threshold cycle time) and relative value method as described by Applied Biosystems and were expressed relative to 18 S rRNA levels.

The genes measured were: CSF-1, MCP-1, GM-CSF, G-CSF, MIP-2, IL-18, IL-1β, IL-5, IL-6, IL-10, p50, p65, TNFα, e-selectin, TGFβ-1, TREM-1, TLR2, TLR4, MMP9, MMP12 and TIMP1.

2.2.9 Myeloperoxidase (MPO) Assay

To determine the levels of MPO activity in the lung tissue of treated mice, a standard MPO assay was performed. Briefly, 50 mg of ground lung tissue (of mice from each treatment group) was resuspended in lmL of MPO Extraction Buffer-MEB (50 mM potassium phosphate (pH 6.0), 0.5% HTA-B [hexa-decyl-trimethyl ammonium bromide, Sigma], and 10 mM EDTA). The tissue was then homogenised with a 20 G needle (20×) and spun at 16,000 g for 20 minutes at 4° C. Supernatant was retained for the assay. 50 μL of the supernatant was then added to 450 μL of freshly prepared reaction buffer (50 mM potassium phosphate (pH 6.0), 0.167 mg/mL O-Da (Fast Blue B, Sigma) and 0.005% H₂O₂) in disposable cuvettes. Absorbance was measured at 460 nm at 1 minute, 2 minutes and 3 minutes. The data was then presented as Absorbance @460 nm per minute, resulting from the decomposition of H₂O₂ and subsequent oxidation of O-Da.

2.2.10 Histology

To ensure consistent morphological preservation of lungs, mice were killed by intraperitoneal anaesthesia (5.6 mg ketamine/1.12 mg xylazine) overdose and then perfusion fixed via a tracheal cannula with 4% formaldehyde at exactly 200 mm H₂O pressure. After 1 h, the trachea was ligated, the lungs were removed from the thorax and immersed in 4% formaldehyde for a minimum period of 24 h. After fixation of the lung tissue and processing in paraffin wax, sections (3-4 μm thick) were cut longitudinally through the left and right lung so as to include all lobes. Sections were stained with hematoxylin and eosin (H&E) for general histopathology.

2.2.11 Metabolic Profiling

As muscle wasting is a co-morbidity of COPD, muscle samples were obtained from these mice for future metabolic profiling. Samples of soleus, tibialis-anterior and gastroc-nemius muscles were obtained from four mice per treatment group and stored at −80° C. for later analysis, such as immunohistochemistry or real-time PCR, at the request of the sponsor.

2.2.12 Statistical Analyses

As data were normally distributed, they are presented as grouped data expressed as mean ± standard error of the mean (s.e.m.); n represents the number of mice. Differences in total BALF cell types and differential counts were determined by one-way analysis of variance (ANOVA) followed by Dunnett post hoc test for multiple comparisons, where appropriate. In some cases, Student's unpaired t-test was used to determine if there were significant differences between means of pairs. All statistical analyses were performed using GraphPad Prism™ for Windows (Version 3.03). In all cases, probability levels less than 0.05 (*P<0.05) were taken to indicate statistical significance.

2.3. Results

2.3.1 Effect of HBP on Inflammatory Cell Number in BALF of Smoke-Exposed Mice

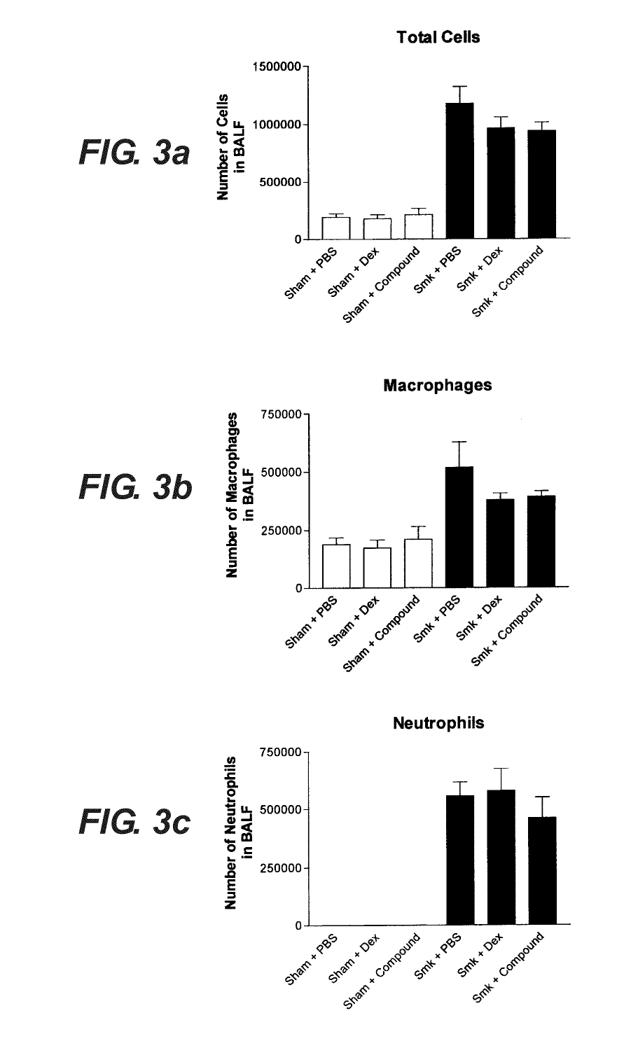

Cigarette smoke exposure caused a significant increase in total viable cell, macrophage, neutrophil and lymphocyte numbers in the BALF of PBS-treated mice, as expected (FIG. 3).

The treatment of sham mice with HBP did not cause an increase in inflammatory cell number compared to PBS-treated sham mice, suggesting that, in healthy mice, HBP did not have an inflammatory effect. In smoke exposed mice, HBP, as with the steroid dexamethasone, did not significantly reduce the numbers of any inflammatory cells. HBP did, however, cause a slight reduction in total viable cells (FIG. 3A), macrophages (FIG. 3B), and neutrophils (FIG. 3C) in the BALF.

2.3.2 Effect of HBP on Protein Levels of TNFα, MIP-2, Keratinocyte Chemoattractant, MMP9 and TIMP-1 in BALF of Smoke-Exposed Mice, as Determined by ELISAs

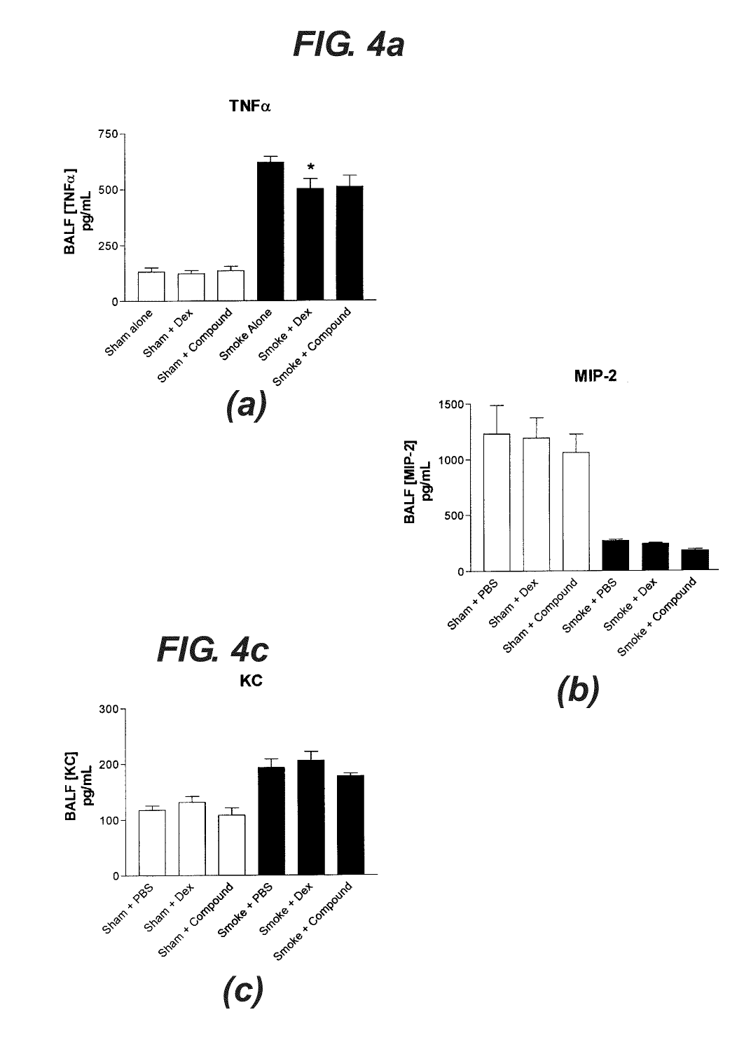

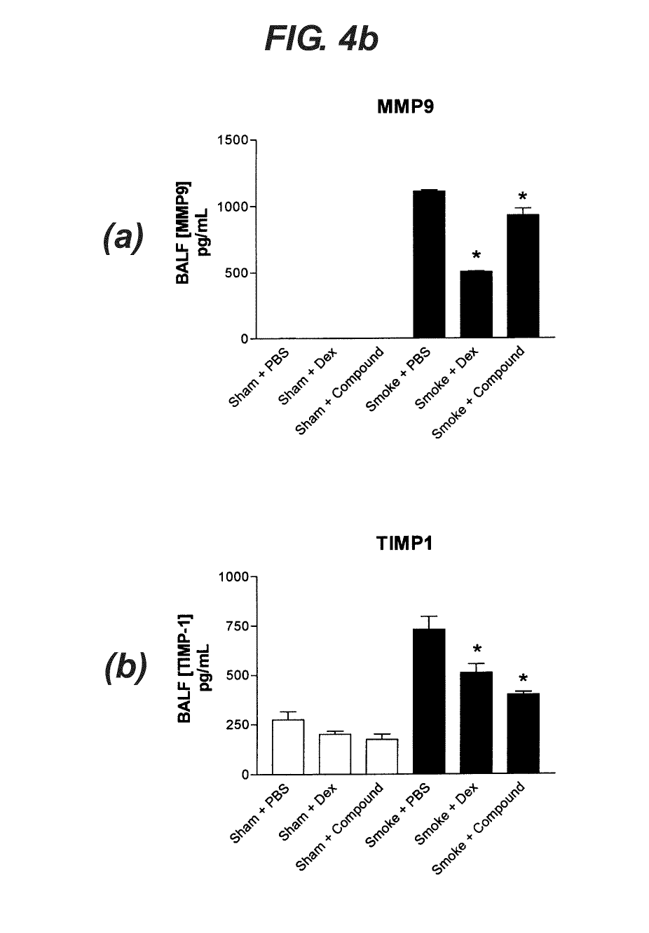

Smoke exposure caused a marked increase in TNFα and keratinocyte chemoattractant protein levels and a marked reduction in MIP-2 protein levels in BALF, 24 hours post-smoke exposure, compared sham animals (FIG. 4A). Smoke also caused a marked increase in MMP9 and TIMP-1 protein levels (FIG. 4B).

Dexamethasone caused a significant reduction in TNFα, MMP9 and TIMP-1 protein levels in the BALF of smoke-exposed mice compared to PBS-treated smoke mice. Dexamethasone did not have an effect on MIP-2 or keratinocyte chemoattractant levels.

HBP did not affect protein levels of TNFα, keratinocyte chemoattractant, MIP-2, MMP9 or TIMP-1 in sham-treated animals. HBP did, however, cause a significant reduction in MMP9 and TIMP-1 protein levels and a marked reduction in TNFα, MIP-2 and keratinocyte chemoattractant levels in the BALF of smoke-exposed animals.

2.3.3 Effect of HBP on Protease Expression and Activity in BALF of Smoke-Exposed Mice, as Determined by Zymography and Protease Assays

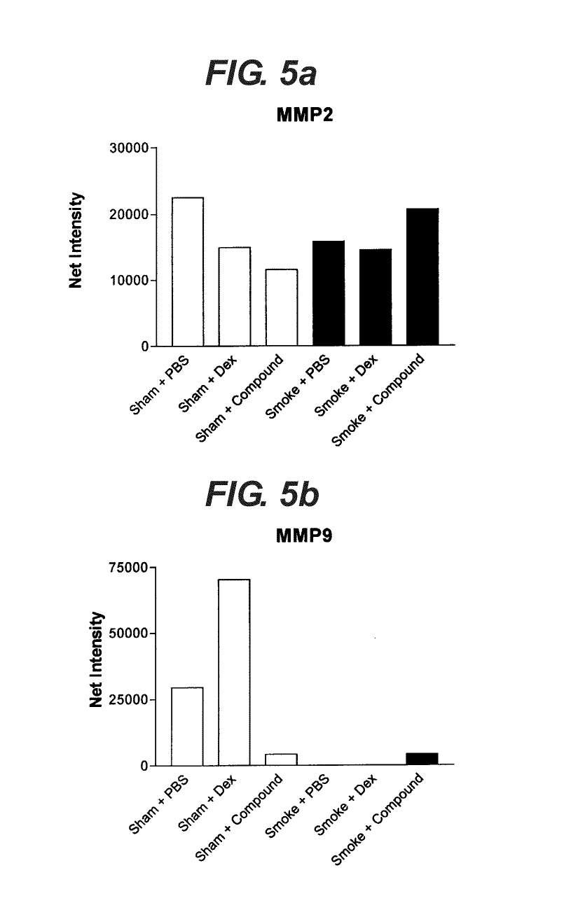

Smoke exposure did not cause any significant changes in MMP2 (FIG. 5a) or MMP9 (FIG. 5b) levels in the BALF compared to sham-treated animals. There was no effect of dexamethasone or test compound on MMP2 or MMP9 levels in the BALF of sham-treated animals or smoke-exposed animals.

There was a slight increase in gelatinase activity in the BALF of smoke-exposed animals (FIG. 5c), and this was slightly reduced with test compound and dexamethasone (more so with the steroid).

2.3.4 Effect of HBP on Expression of Inflammatory Cytokines in the Lung Tissue of Smoke-Exposed Mice, as Determined by Real-Time PCR (Inflammatory Signature Card)

Smoke caused a marked increase in expression levels of all inflammatory cytokines in lung tissue compared to sham-treated animals.

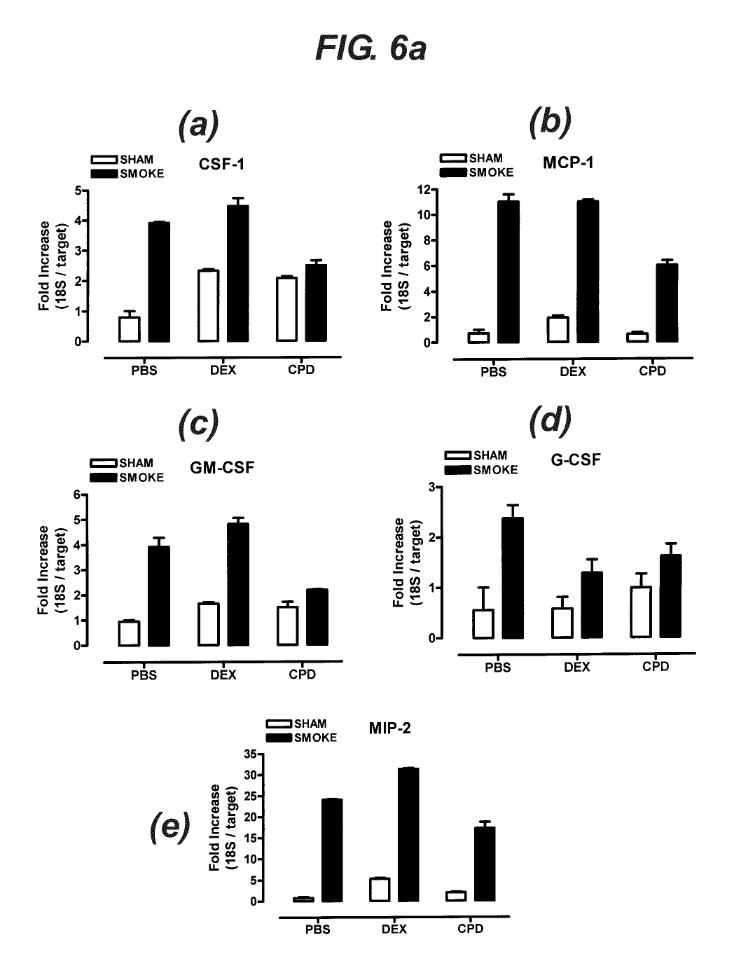

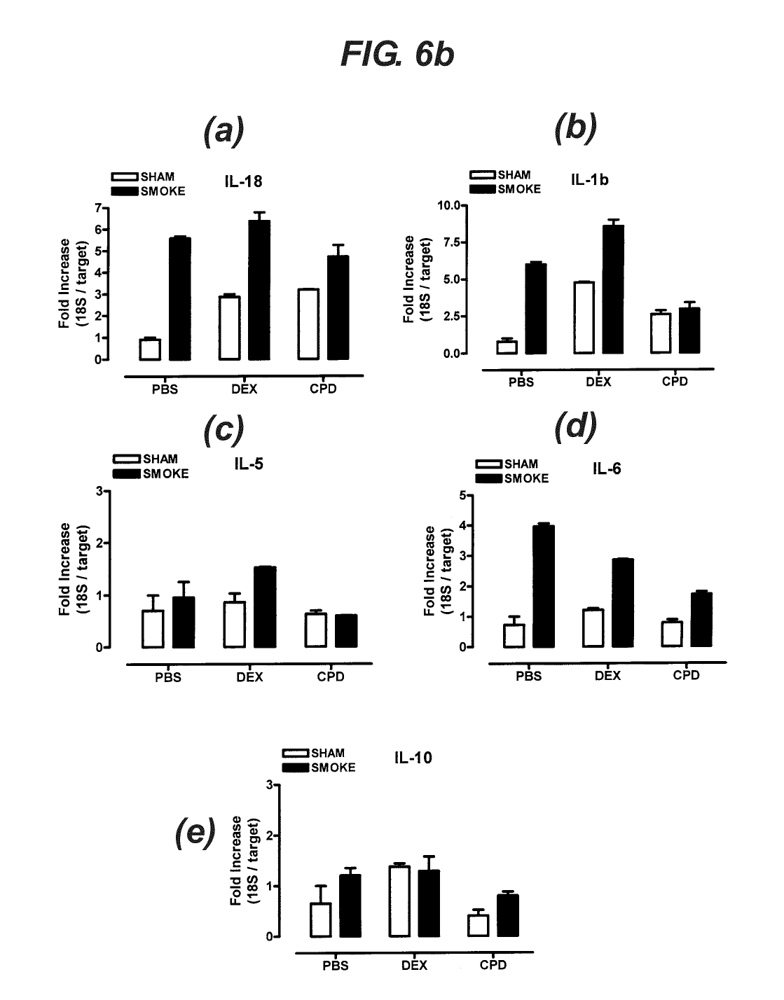

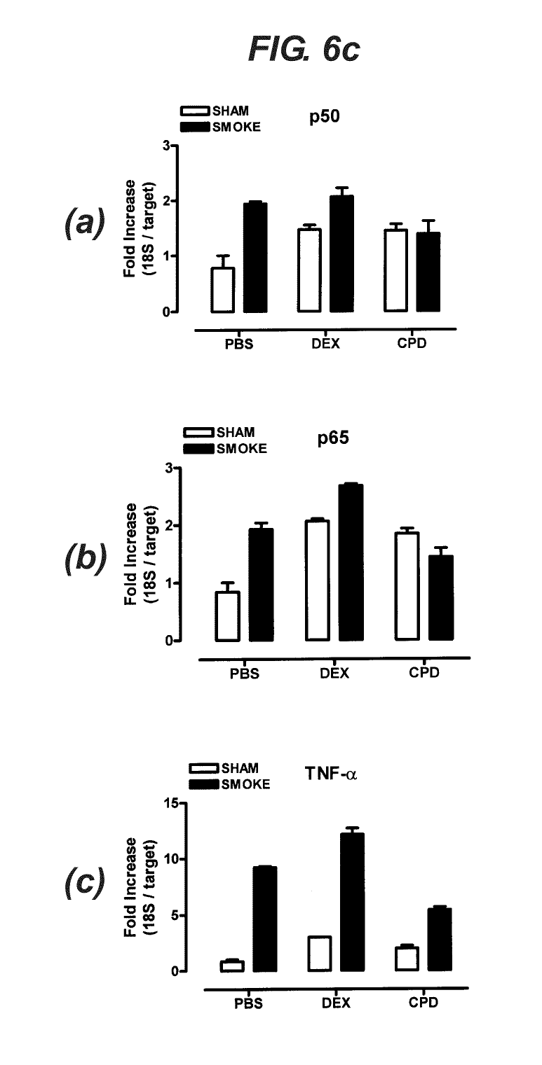

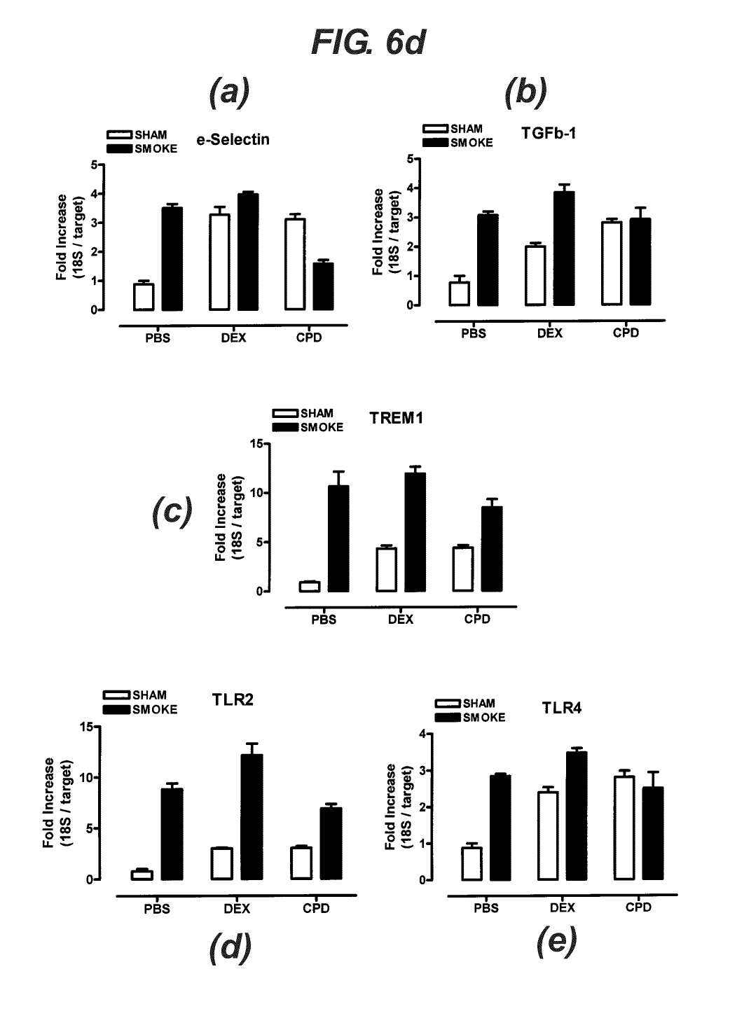

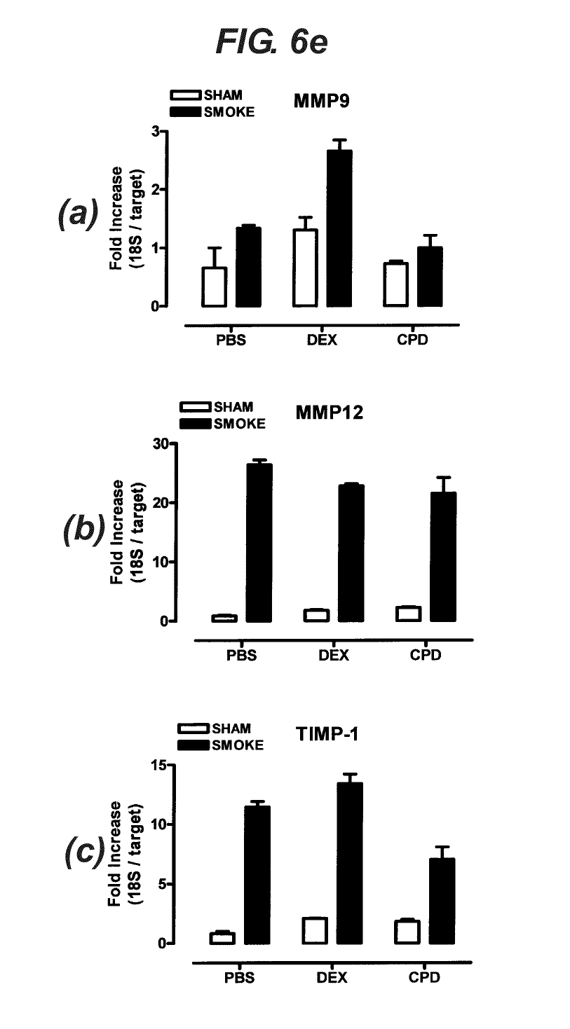

In sham animals, dexamethasone caused increases in CSF-1 and MIP-2 (FIG. 6A), IL-18 and IL-1β (FIG. 6B), p50, p65 and TNFα (FIG. 6C), e-selectin, TGFβ, TREM1, TLR2 and TLR4 (FIG. 6D), and MMP9 (FIG. 6E) in lung tissue. HBP caused increases in IL-18 (FIG. 6B), p50 and p65 (FIG. 6C), e-selectin, TGFβ, TREM1, TLR2 and TLR4 (FIG. 6D) in the lung tissue of sham-treated mice.

Dexamethasone caused a reduction in G-CSF (FIG. 6A) and IL-6 (FIG. 6B), and an increase in MMP9 (FIG. 6E) expression levels in the lung tissue of smoke-exposed mice compared to PBS-treated smoke-exposed mice. HBP caused a reduction in CSF-1, MCP-1, GM-CSF, G-CSF and MIP-2 (FIG. 6A), IL-1β, IL-5, IL-6 and IL-10 (FIG. 6B), p65 and TNFα (FIG. 6C), TLR2, TREM-1 and e-selectin (FIG. 6D), and TIMP-1 (FIG. 6E) relative expression levels in the lung tissue of smoke-exposed mice compared to PBS-treated smoke-exposed mice.

2.3.5 Effect of HBP on Levels of Myeloperoxidase (MPO) in Lung Tissue of Smoke-Exposed Mice

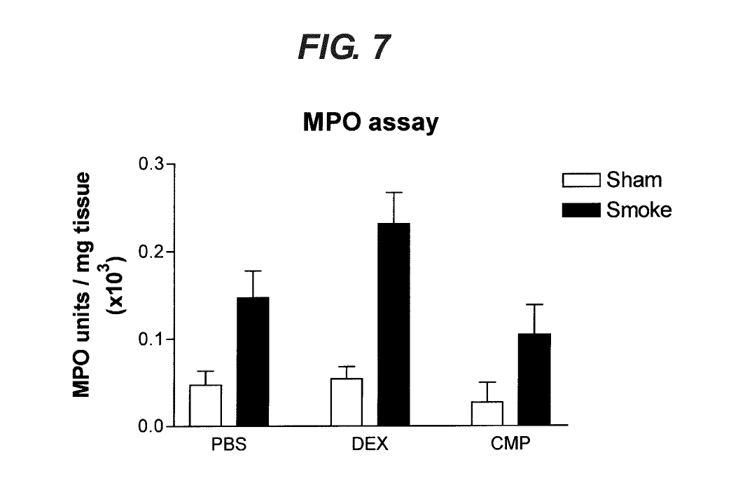

Neither dexamethasone nor HBP had any effect on MPO levels in sham animals (FIG. 7). Cigarette smoke exposure caused a marked increase in MPO levels in lung tissue, which was slightly elevated by subsequent dexamethasone exposure. MPO levels were markedly reduced by HBP.

2.3.6 Effect of HBP on Inflammatory Cell Recruitment Into The Lung Tissue of Smoke-Exposed Mice, As Determined by Histological Analyses

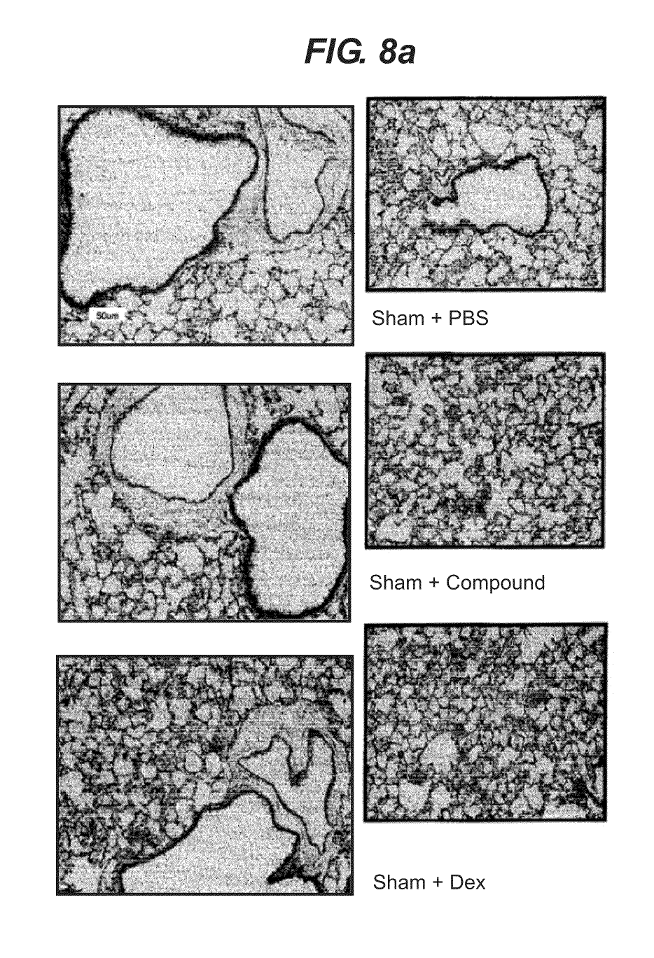

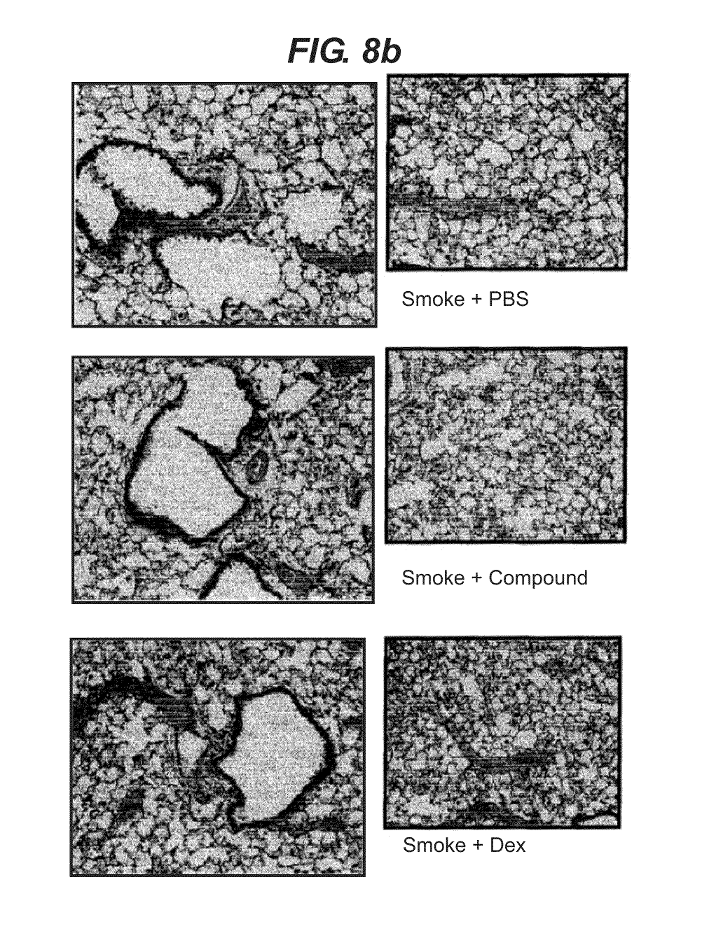

In sham-treated animals (FIG. 8A), HBP had no effect on the appearance of the lung, while dexamethasone caused a slight increase in the inflammation induced by PBS instillation in the lungs.

Cigarette smoke exposure caused a mild lung inflammation recognized by the accumulation of mononuclear cells and some PMNs in the perivascular-peribronchial space and in the alveoli. Dexamethasone did not reduce smoke-induced inflammation, and was associated with an increased intensity of inflammation in the perivascular-peribronchial space and in the alveoli. HBP caused a slight suppression of cellular infiltration into these compartments. However, neutrophils were still evident.

### 2.4. CONCLUSION

The steroid, dexamethasone, slightly exacerbated the mild inflammation caused by vehicle in sham animals, as evident by an elevation in cytokine and protease levels and a slightly more pronounced inflammation as determined by histology. In contrast, HBP did not cause any inflammation in the sham mice.

Cigarette smoke exposure caused a marked increase in inflammatory cells and cytokines in the BALF, increased expression of cytokines and myeloperoxidase in lung tissue, and the influx of inflammatory cells into the airways as determined by histology, and consistent with previous reports [Vlahos, R., et al., Modeling COPD in mice. Pulm Pharmacol Ther, 2006. 19(1): p. 12-7].

The treatment of smoke-exposed mice with dexamethasone had limited efficacy. There was no reduction in BALF inflammatory cell number and, while there was a reduction in BALF protein levels of TNFα, MMP9 and MIP2 and tissue levels of G-CSF and IL-6, there were many other markers of inflammation that were not reduced by steroid treatment. Also, dexamethasone caused a marked increase in myeloperoxidase levels in smoke-exposed animals and appeared to slightly worsen inflammation in the histology preparations, all suggesting that the steroid is of limited utility in this model of lung inflammation. It is known that glucocorticosteroids can increase neutrophil numbers by suppressing apoptosis.

Conversely, HBP caused a slight reduction in total viable cells, macrophages and neutrophils in the BALF of smoke-exposed mice, and caused a marked reduction in a broad range of inflammatory markers, including pro-inflammatory interleukins: IL-1β, IL-5 and IL-6, chemoattractant factors: MIP-2, GM-CSF and MCP-1, and adhesion molecules: e-selectin and TREM-1. The HBP also caused a marked reduction in protein and expression levels of proteases, decreased lung MPO levels and a slight reduction in smoke-induced inflammation determined by histology.

These findings suggest that the HBP suppressed inflammation, especially neutrophil accumulation and activation, more effectively than dexamethasone. This compound appears to have a demonstrable anti-inflammatory effect in this model. This anti-inflammatory profile of the HBP as seen with the data inflammatory gene signature card may be useful in treating the long-term effects in COPD.

### Example 3

### Effect of HBP on Histamine Stimulated Eosinophil shape Change

The mechanism of action of HBP is believed to be the binding of histamine with a greater affinity than membrane bound histamine receptors, thus preventing activation of any of the histamine receptor sub-families of which, to date H1, H2, H3 and H4 receptors have been recognised.

It is believed that the H4 receptor contributes to late phase or chronic inflammation (Daugherty, 2004, Histamine H4 antagonism: a therapy for chronic allergy? Br J Pharmacol, 142, 5-7; Dunford et al., 2006, The histamine H4 receptor mediates allergic airway inflammation by regulating the activation of CD4+ T cells. J Immunol, 176, 7062-70; Fung-Leung et al., 2004, Histamine H4 receptor antagonists: the new antihistamines? Curr Opin Investig Drugs, 5, 1174-83; Jablonowski et al., 2004, The histamine H4 receptor and potential therapeutic uses for H4 ligands. Mini Rev Med Chem, 4, 993-1000; Repka-Ramirez, 2003, New concepts of histamine receptors and actions. Curr Allergy Asthma Rep, 3, 227-31; Schneider et al., 2002, Trends in histamine 20 research: new functions during immune responses and hematopoiesis. Trends Immunol, 23, 255-63; Xie et al., 2005, Roles of histamine and its receptors in allergic and inflammatory bowel diseases. World J Gastroenterol, 11, 2851-7).

Attenuation of H4 receptor activity has been demonstrated to have anti-inflammatory properties in several animal models of late phase inflammation (Takeshita et al., 2004, Critical role of L-selectin and histamine H4 receptor in zymosan-induced neutrophil recruitment from the bone marrow: comparison with carrageenan. J Pharmacol Exp Ther, 310, 272-80; Takeshita et al., 2003, Critical role of histamine H4 receptor in leukotriene B4 production and mast cell-dependent neutrophil recruitment induced by zymosan in vivo. J Pharmacol Exp Ther, 307, 1072-8; Thurmond et al., 2004, A potent and selective histamine H4 receptor antagonist with anti-inflammatory properties. J Pharmacol Exp Ther, 309, 404-13; Varga et al., 2005, Inhibitory effects of histamine H4 receptor antagonists on experimental colitis in the rat. Eur J Pharmacol, 522, 130-8).

The H4 receptor induces late phase inflammation by several mechanisms including upregulation of pro-inflammatory cytokine release (Gantner et aL, 2002, Histamine h(4) and h(2) receptors control histamine-induced interleukin-16 release from human CD8(+) T cells. J Pharmacol Exp Ther, 303, 300-7), down-regulation of IL-12 release (Gutzmer et al., 2005, Histamine H4 receptor stimulation suppresses IL-12p70 production and mediates chemotaxis in human monocyte-derived dendritic cells. J Immunol, 174, 5224-32), regulation of T cells (Dunford et al., 2006, supra), mast cell chemotaxis (Hofstra et al., 2003, Histamine H4 receptor mediates chemotaxis and calcium mobilization of mast cells. J Pharmacol Exp Ther, 305, 1212-21), activation of dendritic cells (Dunford et al., 2006, supra; Gutzmer et al., 2005, supra) and eosinophil chemotaxis and shape change (Buckland et al., 2003, Histamine induces cytoskeletal changes in human eosinophils via the H(4) receptor. Br J Pharmacol, 140, 1117-27; Ling et al., 2004, Histamine H4 receptor mediates eosinophil chemotaxis with cell shape change and adhesion molecule upregulation. Br J Pharmacol, 142, 161-71; Nakayama et al., 2004, Liver-expressed chemokine/CC chemokine ligand 16 attracts eosinophils by interacting with histamine H4 receptor. J Immunol, 173, 2078-83; O'Reilly et al., 2002, Identification of a histamine H4 receptor on human eosinophils--role in eosinophil chemotaxis. J Recept Signal Transduct Res, 22, 431-48; Rothenberg et al., 2006, The eosinophil. Annu Rev Immunol, 24, 147-74).

Several of these activities are known however to respond to activation of other histamine receptors in addition to the H4 receptor and in some cases the activation of more than one receptor is needed to achieve maximal response. Release of IL-16 from human CD8+ lymphocytes is in part induced by H2 receptor activation (Gantner et al., 2002, supra). H1 receptor activation contributes to T cell regulation (Dunford et al., 2006, supra). Pruritic responses in mice can be attenuated by blockade of either H1 receptor or H4 receptor (Bell et al., 2004, Involvement of histamine H4 and H1 receptors in scratching induced by histamine receptor agonists in Balb C mice. Br J Pharmacol, 142, 374-80). Th2 polarisation of dendritic cells can be influenced by H1 receptor in addition to H4 receptor (Mazzoni et al., 2001, Histamine regulates cytokine production in maturing dendritic cells, resulting in altered T cell polarization. J Clin Invest, 108, 1865-73).

One of the few late phase inflammatory activities that is believed to be purely H4 receptor dependent is histamine stimulated eosinophil shape change and it has been demonstrated that blockade of other histamine receptors has no effect on this (Buckland et al., 2003, supra; Ling et al., 2004, supra). Influx of eosinophils characterise the late phase of allergic inflammation due to IgG activation of mast cells. In order to facilitate migration through the walls of capillaries by diapedesis eosinophils change shape to a more elongated form. A number of different mediators are known to be capable of inducing this shape change including eotaxin, eotaxin-2 and MIP-1α (Sabroe et al., 1999, Differential regulation of eosinophil chemokine signaling via CCR3 and non-CCR3 pathways. J Immunol, 162, 2946-55). Shape change is caused by influx of calcium ions triggering polymerisation of actin fibrils which draw opposing sides of the cell membrane together. When histamine stimulated, the process is believed to be entirely H4 receptor dependent.

Histamine-induced eosinophil shape change and its complete abolition by H4 receptor blockade can be demonstrated by flow cytometry using the gated autoluminescence forward scatter (GAFS) assay (Buckland et al., 2003, supra; Ling et al., 2004, supra). In the experiment below, it is shown that the HBP of the invention can achieve the same effect without receptor blockade.

Materials and Methods:

Cells were harvested using the method of Buckland et al. (Buckland et al., 2003, supra). Eosinophils were purified from peripheral blood samples obtained from normal and atopic volunteers. Thirty five ml of whole blood was containing eosinophils and neutrophils was taken into 4.4 ml of 3.8% tri-sodium citrate and centrifuged at 300 g for 20 minutes. Plasma was discarded and remaining cells resuspended in 0.6% dextran in saline. After separation of red blood cells by sedimentation for 30 minutes the leukocytes were layered over Histopaque® and centrifuged for 25 minutes. Mononuclear cells were isolated as a separate band on the Histopaque® and discarded. Remaining granulocytes were resuspended and contaminating red blood cells lysed by hypotonic shock. The granulocytes were then washed, counted and resuspended in PBS buffer (PBS without Ca⁺⁺ Mg⁺⁺, containing 0.1% wv⁻¹ BSA, 10 mM glucose, 10 mM HEPES) at 1×10⁷ cells/ml⁻¹.

Eosinophil shape change was assayed using the method of Sabroe et al. (Sabroe et al., 1999, supra). Leukocytes were stabilised for 30 minutes at room temperature and were then centrifuged and resuspended in PBS buffer (PBS with or without Ca⁺⁺ Mg⁺⁺ or antagonists as required, containing 0.1% wv⁻¹ BSA, 10 mM glucose, 10 mM HEPES) at 1×10⁷ cells/ml⁻¹ and incubated for a further 15 minutes. Cells were stimulated with agonists diluted in PBS buffer with or without Ca⁺⁺ Mg⁺⁺ for 4 minutes at 37° C. before fixation with CellFix® at 4° C. to maintain cell shape and sample fluorescence measured by flow cytometry using a Becton Dickinson FACSCaliber. Eosinophils were identified and gated by their natural autofluorescence, which is greater than that of neutrophils, in the FL-2 channel. Data were obtained for 500 events within the high fluorescence gated region identified as eosinophils. Results are expressed as a percentage increase in forward scatter (FSC) compared to unstimulated cells.

Histamine dichloride 1.0 or 0.5 μM (Sigma) was used to stimulate the granulocytes. The specific H4 receptor antagonist JNJ10191584, the H2 receptor antagonist cimetidine and the Hi receptor mepyramine were obtained from Tocris Cookson Ltd. UK, the specific H4 receptor antagonist JNJ7777120 and the H3/H4 antagonist thioperamide were obtained from Sigma Aldrich, UK.

Results:

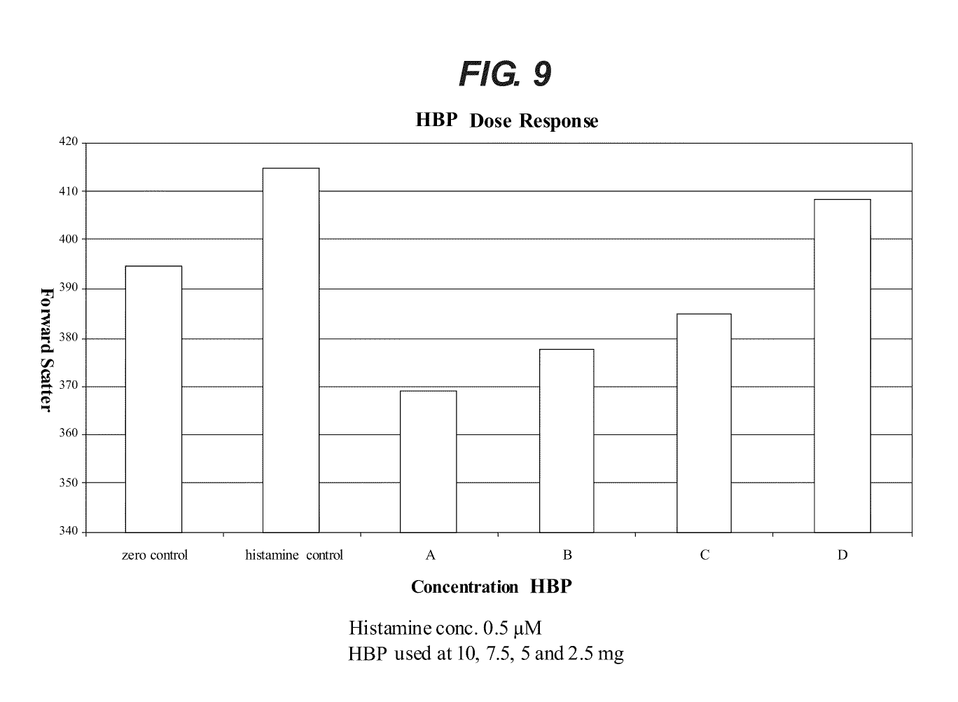

Using pooled eosinophils from normal and atopic subjects mean increase in FSC of ≅ 30% above baseline was observed following histamine stimulation. Incubation of the cells with HBP dose dependently abolished FSC and at highest doses reduced it below baseline (unstimulated) levels by≅50% (FIG. 9).

JNJ7777120, JNJ10191584 and thioperamide all dose-relatedly reduced histamine stimulated FSC but did not reduce it below baseline (unstimulated) levels (data not shown).

### CONCLUSION

The histamine binding protein HBP abolishes histamine stimulated shape change in human eosinophils in dose-related fashion in the gated autoluminescence forward scatter (GAFS) assay. As this has been shown to be dependent on the histamine H4 receptor it is concluded that HBP prevents activation of this receptor.

At maximum doses HBP reduced shape change 50% below baseline (unstimulated) levels. In contrast, specific H4R antagonists and the H3/H4 receptor antagonist thioperamide reduced it to baseline and no further, in accordance with the findings of previous workers in the field (Buckland et al., 2003; Ling et al., 2004).

As the significance of histamine stimulated shape change is to increase the inflammatory potential of eosinophils it is further concluded that a reduction below baseline levels as caused by HBP is indicative of enhanced anti-inflammatory activity compared with H4 receptor blocking agents.

### Example 3

### Stability of HBP

Samples of HBP lot no. P01105B 0.63 mg/ml and lot no. P01105E 5.0 mg/ml were tested after storage at 4° C. and 25° C./60% RH for 52 weeks.

The following assays were performed: purity/identity by SDS-PAGE, potency by the HUVEC bioassay and aggregation by sedimentation velocity.

Assessment of Purity and Identity by SDS-PAGE:

Precast gels 4-20% were prepared for gel electrophoresis. The test items were run under reducing and non-reducing conditions at indicated final concentrations of 0.30 mg/ml and 0.15 mg/ml. The reference standard was run at 0.60, 0.30 and 0.15 mg/ml. The samples were heated for 5 min at 70° C. and then put on ice. 10 μl standard and 10 μl sample per lane were loaded per lane and the gel was run at 100V for 120 min until the dye front was about 1.5 cm from the bottom of the gel. Then, the gel was stained with Coomassie Blue and an image was taken with a CCD camera.

The gels were analysed by the GelScan 5 Pro BioSciTec (2001) software. Bands obtained on the gel were analysed in relation to the standard bands run in the gel. Molecular weights were related to the list of the molecular weights standards. The main band was identified.

Net intensity of the bands was determined by using the automatic mode with background subtraction. The net intensity of the main band represents the purity of the test item.

Assessment of Potency by HUVEC Bioassay.

The assay is based on the histamine-dependent increase of IL-6 production in TNFα activated human umbilical vein endothelial cell (HUVEC) monolayers. Removal of histamine by histamine scavenging compounds is expected to have an influence on IL-6 production, Thus, the potency of the HBP can be correlated with the IL-6 production of HUVECS.

HUVECS were cultured in fibronectin-coated 96-well tissue culture plates. After reaching confluence, serum concentration of the culture medium was reduced to 2% and the cells were stimulated with TNFα and histamine for another 18 hours to induce production of IL-6 which was quantified by ELISA. The potency of HBP to scavenge histamine was studied by coincubation of the HUVECs with different concentrations of the HBP test items.

The reference standard HBP batch 074 was assayed in parallel. All test items were assayed in duplicates or triplicates. To ensure reliability of the results, each assay was required to pass two quality specifications:

i) Histamine Effect on HUVECs

The enhancement of IL-6 production by histamine was calculated using the formula:

Histamine effect (%)=(A×100/B)−100

wherein A is mean absorbance in cultures stimulated with TNFα and histamine and B is mean absorbance in cultures stimulated with TNFα.

The histamine effect must be >30%

ii) Performance Qualification

In order to calculate IC50 values, each test item was tested for its maximal inhibitory effect on IL-6 production. The performance is expressed as % maximal inhibition by HBP and was calculated using the following formula:

Maximal inhibition by HBP (%)=100−(A×100/B−100)

wherein A is the mean value of lowest absorbance found within the titration experiment of a given sample of HBP and

B is the mean absorbance in cultures stimulated with TNFα.

% maximal inhibition by HBP must be 100±15%.

Calculation of the histamine effect and the performance quantification was done using the OD values from the IL-6 ELISA experiments.

IC₅₀ values were calculated in samples meeting these criteria. For the calculation of the IC₅₀ values, OD values from the IL-6 ELISA were normalised on minimum (set to 0) and maximum (set to 1), after backgrounds subtraction. This procedure allows a comparison of different experiments performed at different time points within the time frame of the stability testing.

Results:

The examination of identity and purity by SDS-PAGE under reducing and non-reducing conditions revealed for the test items 100% purity and a molecular weight that was conform with HBP reference 8.3 mg/ml.

For determination of IC₅₀, the HUVEC assay had to pass the criteria “Histamine effect” and “performance qualification”. All test items passed these criteria. In the subsequent evaluation of the IC₅₀ values, the test items were compared to the HBP reference (8.3 mg/ml). The criteria for acceptance was “conformity to reference standard” (100±15%). All test items passed the acceptance criteria.

Additional examination of HBP aggregation at 25° C./60% RH by sedimentation velocity revealed no significant aggregation of HBP lot no. P01105B 0.63 mg/ml) and lot no. P01105E 5.0 mg/ml.

In summary, the data demonstrate the stability of the HBP at 4° C. and 25° C./60% RH for 52 weeks.

### Example 4

### Quantitative Whole Body Autoradiography Studies of HBP Distribution Following Administration to Rats

The distribution of HBP was investigated in the rat, using ¹²⁵I-labeled test substance. Experiments were conducted at a dose level of 15 μg/kg.

Plasma Pharmacokinetics:

A summary of the mean pharmacokinetic parameters of total radioactivity observed following intravenous administration of ¹²⁵I-HBP are given in the following table:

| Parameter | Total radioactivity |
|---|---|
| Cₘₐₓ (ng equiv./mL) | 3.994 |
| Tₘₐₓ (hours) | 0.5 |
| AUC₀₋₄₈ (ng equiv/mL.h) | 28.1 |
| AUC_{inf} (ng equiv./mL.h) | 28.5 |
| t_{1/2} (hours) | 7.83 |

Cₘₐₓ=maximum plasma concentration

Tₘₐₓ=time of maximum plasma concentration

AUC₀₋₄₈=area under curve from time of dosing to last measurable concentration

AUC_{inf}=area under curve from time of dosing extrapolated to infinity

t_{1/2}=apparent terminal elimination of half life

Tissue Distribution

Following an intravenous dose of ¹²⁵I-HBP, concentrations of radioactivity in tissues were measured using whole body autoradiography procedures and a summary of the notable data is given in the following table.

| Tissue | 0.5 hours | 2 hours | 24 hours |
|---|---|---|---|
| Brain | BLQ | BLQ | BLQ |
| Blood | 0.012 | 0.002 | BLQ |
| Kidney | 0.675 | 0.202 | 0.001 |
| Liver | 0.028 | 0.006 | 0.001 |
| Thyroid gland | 0.038 | 0.196 | 0.850 |
| Urinary bladder | 0.386 | 0.130 | 0.005 |

Results expressed as μg equivalents/g

BLQ=Below limit of quantification (<0.001 μg equivalents/g)

The results indicate that after dosing, absorbed radioactivity was extensively distributed throughout all tissues. Radioactivity concentrations in the brain were at levels below the limit of quantification at all time points which would suggest that there is no transfer of HBP across the blood-brain barrier.

Maximal concentrations in tissues were generally observed at 0.5 hours, the first sampling time point.

Greatest concentrations of radioactivity were observed in the kidney and urinary bladder. After 168 hours, radioactivity in all tissues had declined with the exception of the thyroid gland. This increase of radioactivity in the thyroid gland is though to be associated with free iodide.

These data demonstrate that HBP administered intravenously is widely distributed in a variety of tissues which is indicative of its utility in therapeutic applications.

1. A histamine binding protein (HBP) comprising the sequence presented in SEQ ID NO:1.

2. An HBP according to claim 1, which consists of the sequence presented in SEQ ID NO:1.

3. An HBP according to claim 1 which binds to histamine with a dissociation constant of less than 10⁻⁷ M.

4. An HBP according to claim 3, which binds specifically to histamine.

5. An HBP according to claim 1, which is stable at room temperature.

6. An HBP according to claim 1, which has a half-life of around 30 hours, as assessed in a mammalian reticulocyte system.

7. A nucleic acid molecule which encodes an HBP polypeptide according to claim 1.

8. A nucleic acid molecule according to claim 7, which comprises the sequence presented in SEQ ID NO:2.

9. A nucleic acid molecule according to claim 8, which consists of the sequence presented in SEQ ID NO:2.

10. A vector comprising a nucleic acid molecule as recited in claim 7.

11. A host cell transformed with a vector according to claim 10.

12. A pharmaceutical composition comprising an HBP according to claim 1.

13. A pharmaceutical composition according to claim 12, formulated in a formulation buffer with PBS.

14. A pharmaceutical composition according to claim 12, formulated as a cream adapted for topical administration.

15. A pharmaceutical composition according to claim 12, formulated as an aerosol.

16. A method of treating a patient suffering from a disease condition in which histamine is implicated by administering an HBP according to claim 1 to the patient in a therapeutically-effective amount.

17. A method according to claim 16, wherein said disease condition is selected from the group consisting of allergies, such as allergic rhinitis, allergic conjunctivitis (including severe allergic conjunctivitis), vernal keratoconjunctivitis (VKC), diffuse lamellar keratitis, infective and non-specific conjunctivitis, keratitis and blepharitis; and disease conditions in which neutrophils are implicated, including adult respiratory distress syndrome (ARDS); infant respiratory distress syndrome (IRDS); severe acute respiratory syndrome (SARS); chronic obstructive airways disease (COPD); cystic fibrosis; ventilator induced lung injury (VILI); capillary leak syndrome; reperfusion injury including injury following thrombotic stroke, coronary thrombosis, cardiopulmonary bypass (CPB), coronary artery bypass graft (CABG), limb or digit replantation, organ transplantation, bypass enteritis, bypass arthritis, thermal injury and crush injury; post-operative inflammation or marginal infiltrates, psoriasis; psoriatic arthropathy; rheumatoid arthritis; Crohn's disease; ulcerative colitis; immune vasculitis including Wegener's granulomatosis and Churg-Strauss disease; alcoholic liver disease; neutrophil mediated glomerulonephritis; systemic lupus erythematosus; lupus nephritis; atherosclerosis; systemic sclerosis; gout; periodontal disease, ocular inflammation including dry eye, Sjogren's syndrome, contact lens associated papillary conjunctivitis (CLAPC), contact lens associated marginal infiltrates, post surgical inflammation including surgery for cataract, glaucoma, corneal transplantation and laser in-situ keratomileusis (LASIK), and shield ulcers.

18. A method for preparing a HBP according to claim 1 which comprises culturing a host cell containing a nucleic acid molecule which encodes an HBP polypeptide according to claim 1 under conditions whereby the protein is expressed and recovering said protein thus produced.

19. (canceled)

20. (canceled)

21. (canceled)

22. A method of treating a patient suffering from a disease condition in which histamine is implicated by administering a pharmaceutical composition according to claim 12 to the patient in a therapeutically-effective amount.

23. A method according to claim 22, wherein said disease condition is selected from the group consisting of allergies, such as allergic rhinitis, allergic conjunctivitis (including severe allergic conjunctivitis), vernal keratoconjunctivitis (VKC), diffuse lamellar keratitis, infective and non-specific conjunctivitis, keratitis and blepharitis; and disease conditions in which neutrophils are implicated, including adult respiratory distress syndrome (ARDS); infant respiratory distress syndrome (IRDS); severe acute respiratory syndrome (SARS); chronic obstructive airways disease (COPD); cystic fibrosis; ventilator induced lung injury (VILI); capillary leak syndrome; reperfusion injury including injury following thrombotic stroke, coronary thrombosis, cardiopulmonary bypass (CPB), coronary artery bypass graft (CABG), limb or digit replantation, organ transplantation, bypass enteritis, bypass arthritis, thermal injury and crush injury; post-operative inflammation or marginal infiltrates, psoriasis; psoriatic arthropathy; rheumatoid arthritis; Crohn's disease; ulcerative colitis; immune vasculitis including Wegener's granulomatosis and Churg-Strauss disease; alcoholic liver disease; neutrophil mediated glomerulonephritis; systemic lupus erythematosus; lupus nephritis; atherosclerosis; systemic sclerosis; gout; periodontal disease, ocular inflammation including dry eye, Sjogren's syndrome, contact lens associated papillary conjunctivitis (CLAPC), contact lens associated marginal infiltrates, post surgical inflammation including surgery for cataract, glaucoma, corneal transplantation and laser in-situ keratomileusis (LASIK), and shield ulcers.
